(19) United States
(12) Patent Application Publication
(10) Pub. No.: US 2024/0335597 A1
(43) Pub. Date: Oct. 10, 2024

(54) IMPLANTABLE OR PARA-CORPOREAL BLOOD FILTRATION AND OXYGENATION DEVICE FOR MAINTAINING HOMEOSTASIS AND METHODS THEREOF

(71) Applicant: SHLOMO GABBAY, BOCO RATON, FL (US)

(72) Inventor: SHLOMO GABBAY, BOCO RATON, FL (US)

(21) Appl. No.: 18/293,931

(22) PCT Filed: Jul. 29, 2022
(86) PCT No.: PCT/US22/38769

§ 371 Date: Jan. 31, 2024

Related U.S. Application Data
(60) Provisional application No. 63/228,225, filed on Aug. 2, 2021.

(51) Int. Cl. A61M 1/16; A61M 1/14

(52) U.S. Cl. CPC A61M 1/1678; A61M 1/1561; A61M 1/1698; A61M 2205/0266

(57) ABSTRACT

Corporeal and extra-corporeal filtration and oxygenation devices include multi-chambered structures having a semi-permeable membrane between two chambers for creating differentials to extract waste from and/or add oxygen to blood flowing through the devices. A bio-modified material covers multiple surfaces of the devices which encounters blood to reduce blood contact with polymeric surfaces. Blood contact with polymeric surfaces causes a chemical cascade which in turn induces an inflammatory response in a patient's body. The filtration and oxygenation devices, and methods of installing and using the devices, reduce the frequency and severity of the inflammatory.

### BACKGROUND OF THE INVENTION

### Field of the Invention

The present invention pertains generally to the reduction and/or prevention of immunological insults typically acquired during medical treatments involving oxygenation or filtration of blood.

The present invention therefore relates to medical devices and methods of using the same for implantation fully or partially within a body of a human patient to filter blood in order to remove waste, toxins, and unwanted fluid without activating an inflammatory response within the body. Additionally, the same medical devices can be fully or partially implanted within the body to oxygenate blood. More specifically, the present invention relates to implantable devices that are able to perform dialysis, cardio-pulmonary bypass, and/or extra-corporeal membrane oxygenation (ECMO) without activating an inflammatory response in the body. Additionally, the present invention includes methods of installing the implantable devices, either fully or partially within the body, along with methods of performing dialysis, cardio-pulmonary bypass, and/or ECMO without activating the inflammatory response in the body.

### Brief Discussion of the Prior Art

Kidney disease in a human includes one or more kidneys that are damaged and cannot filter blood properly. Improper blood filtration leads to waste to build up in a human body. This build-up of waste in the body can have widespread impact on various organs and increases the risk for stroke or cardiac arrest. End-stage renal disease (ESRD) involves complete, permanent kidney failure. ESRD can only be treated with a kidney transplant or dialysis.

The risk factors for kidney disease vary across genetic predisposition to environmental exposures, and include diabetes mellitus, hypertension, and a family history of kidney failure. In 2021, the Centers for Disease Control and Prevention (CDC) estimated that 1 in 7 adults in the United States have chronic kidney disease (CKD), which equates to roughly 37 million people. See Centers for Disease Control and Prevention. Chronic Kidney Disease in the United States, 2021. Atlanta, GA: US Department of Health and Human Services, Centers for Disease Control and Prevention; 2021. CKD means that a patient's kidneys are damaged but have not yet failed completely to result in ESRD.

The 2018 USRDS annual data report estimated that 750,000 people per year suffer from kidney failure in the United States alone, with 2 million people suffering from kidney failure worldwide. In the United States, patients with ESRD account for 1% of the U.S. Medicare population, but account for 7% of the Medicare budget. More than 100,000 patients in the United States are on the kidney transplant list, but there were just over 21,000 donor organs available for transplant in 2017. Alarmingly, the need for donor kidneys in the United States to combat ESRD is rising at 8% per year. See 2018 USRDS annual data report: Epidemiology of kidney disease in the United States. National Institutes of Health, National Institute of Diabetes and Digestive and Kidney Diseases, Bethesda, M D, 2018.

When treating ESRD, the only alternative to a kidney transplant is regular dialysis treatment. Dialysis largely replicates the functions of the kidneys in patients with CKD or ESRD. Dialysis, including common dialysis treatment types, hemodialysis and peritoneal dialysis, takes over the key filtration tasks of the kidneys, such as removing waste materials, toxins, excess salt, and fluids from the body.

However, in view of current understandings of kidney treatment, medical knowledge, and available technology, dialysis treatment cannot entirely replace all the functions of the kidneys. This means that patients undergoing dialysis almost always need to take certain medications regularly. Such medications may include antihypertensive treatments, drugs for lowering phosphate levels in the blood, and vitamins and drugs that boost the production of red blood cells to prevent anemia. Out of many in the field of kidney treatment, a goal would be to reduce the number and type of medications necessary to complement dialysis by obviating the extravasation of blood and contact with synthetic surfaces that are reactive with blood.

Hemodialysis is the most common form of dialysis in the United States. It involves removing blood from an access point in a vein, then cleaning the blood of toxins and excess fluids through a filter device before returning it to the body. The filter device is called a dialyzer. The dialyzer is an artificial filter having fine fibers. The fine fibers are hollow with microscopic pores in the wall, also known as semi-permeable dialysis membrane. To remove toxins during hemodialysis, a special dialysis-fluid flows through the filter, and bathes the fibers from the outside, while the blood flows through the hollow fiber. Due to the semi-permeable dialysis membrane, toxins, urea and other small particles can pass through the membrane, but the blood cells cannot. Hemodialysis is meant to replicate the function of the kidneys, but it is an inexact science. If the speed of the procedure is too aggressive, or the individual being treated does not adhere to the proper dietary or fluid restrictions, the homeostasis, or internal balance, of the body chemistry can be thrown off, causing side effects and complications. The creation of an artificial access point in the body also poses a risk in that the closed vascular system is now open. Infection is the most common concern, but not the only one.

Peritoneal dialysis involves inserting a catheter into the lining of the patient's abdomen, or the peritoneum. The catheter is attached to an external bag of fluid. This cleansing fluid flows from the bag and into the peritoneum through the catheter. The peritoneum acts as a filter through which waste products flow into the cleansing fluid. After a given amount of time, the cleansing fluid, now full of waste product, is removed via the catheter to a separate bag and discarded. Peritoneal dialysis allows for greater lifestyle flexibility than hemodialysis, as it can be performed at home and requires a less restrictive diet. However, peritoneal dialysis also requires greater independence and is only practical if the patient is competent to care for himself or herself or has at-home health care assistance.

Various complications are associated with vascular access in patients who are on hemodialysis and are associated with abdominal catheters in patients using continuous ambulatory peritoneal dialysis (CAPD). These vascular access complications are similar to those seen in any patient with a vascular surgical procedure (e.g., bleeding, local or disseminated intravascular infections, and vessel occlusion).

Such complications include hyperkalemia, hypocalcemia, hyponatremia, and hypermagnesemia. Possible neurologic complications include headache, dialysis dementia, dialysis disequilibrium syndrome, Wernicke's encephalopathy, and stroke, which can occur either directly or indirectly in relation to hemodialysis. In short, dialysis can cause a total disturbance of the electrolytes in the patient's body. This is due either to removal of the blood outside the body during hemodialysis, or to contact with plastic tubing common in either hemodialysis or peritoneal dialysis. Such disturbances, further referred to as a non-natural event, involve anti-homeostatic processes which disrupt the body's normal range of internal chemical balance to produce immunological insults, which are various immunological responses to the non-natural events.

Such immunological insults are also seen in patients undergoing cardio-pulmonary bypass and ECMO. Both of these treatments involve oxygenation of a patient's blood using an extra-corporeal oxygenator, or a machine outside of the body that adds oxygen to blood. Cardio-pulmonary bypass is the procedure used during surgery, specifically open-heart surgery, which allows the heart and lungs to lie still while a surgeon operates on the patient. Deoxygenated blood is passed from a blood vessel through tubing to the heart-lung machine, which pumps the blood through an oxygenator. This oxygenated blood is then reintroduced via tubing into the patient through a blood vessel during surgery.

ECMO involves the same concept but is performed while the heart and the lungs are still operational. A pump removes deoxygenated blood from the patient's body, pumps it to an oxygenator, and then pumps oxygenated blood back into the patient's body. While the patient's heart and lungs are still operational, ECMO reduces stress on these organs to allow them to rest, recuperate, and heal. Application of ECMO is uneven across the medical field, but it is commonly applied to patients recovering from heart attack, an infection in the lungs, and recently to COVID-19 infections. All of these illnesses include severe stress on the heart and/or lungs.

Cardio-pulmonary bypass and ECMO are not readily available to most patients. These procedures are only found in larger hospitals with heart programs, i.e., a dedicated medical staff for performing heart surgeries and transplants. Neither treatment can currently be performed at home. Further, both treatments are stressful on the body and cannot be performed in patients with certain pre-existing conditions, including advanced age, morbid obesity, severe immunocompromised status, and advanced heart and/or lung failure.

Both the kidneys and the lungs are biologically meant to be blood-contacting structures and surfaces. Blood cells function properly when coming into contact with such blood-contacting surfaces. As such, whole organ transplant, i.e., removing and replacing a damaged kidney or lung with an otherwise properly functioning kidney or lung from another person, is the best treatment for kidney or lung failure. Such treatment is obviously limited in availability, and other modes of treatment, often including plastic tubing or other devices made of polymers, have been developed. However, blood-contacting structures and surfaces did not evolve to naturally contact with the plastics. Unfortunately, plastics are highly prevalent in dialysis, cardio-pulmonary bypass, and ECMO, which are the next best alternatives to a transplant or necessary treatments in preparation of, or during, transplant.

It has been found that blood contact with plastic surfaces causes an immunological insult. In 1989, a study was conducted by the inventor, Dr. Gabbay, on sheep to test the feasibility of using absorbable material as a pericardial substitute, to compare biological material to biodegradable material when used as pericardial substitutes with and without cardio-pulmonary bypass (CPB), and to observe frequency and severity of calcification with a biological pericardial substitute. While this study focused on cardiovascular structures, the results of the study showed that CPB caused adhesion between the pericardial substitute and the heart, along with causing a thick layer of fibrin to form along the epicardium. This fibrin layer severely prohibits reoperation, i.e., a second or subsequent surgery. Calcification of collagen, along with inhibition of collagen replacement, and sever lesions were also seen in the CPB group. The study concluded that these results are dependent on operating conditions and not on species differences between humans and other animals. See Gabbay, S., Guindy, A. M., Andrews, J. F., Amato, J. J., Seaver, P., and Khan, M. Y. New Outlook on Pericardial Substitution After Open Heart Conditions. Ann Thorac Surg. 1989; 48:803-12.

There is evidence dating back more than 30 years that removing blood from the body and putting it into contact with plastics causes unwanted biological consequences when that blood is returned to the body. While the referenced study dealt with removal and return of blood for oxygenation purposes, the same principle applies to removal and return of blood for filtration purposes. The body reacts severely to this non-natural event and activates the immune system in response to foreign interference with its tissue, causing an immunological insult. This activation of the immune system is actually an auto-immune disease. If it occurs twice a week, as is common with dialysis treatment, it will shorten the life of the patient. Even short-term immunological insults, such as those prompted by CPB and ECMO, can be a major threat to patient health if the patient is already under severe stress, such as during open-heart surgery or recovery from heart attack, lung infection, or COVID-19 infection. Such immunological insults are a major threat to patient survivability and quality of life.

Studying the history of blood oxygenators used in CPB, it has been realized that the non-natural event of blood contacting with the plastic polymers is the culprit for the immunological insult. Thus, silicone coating has been used with some improvements, but the problem has persisted. Polyethylene and polypropylene have been used without significant improvement. Human bodies are not naturally compatible with silicone, polyethylene, and polypropylene, especially as blood-contacting surfaces.

Further studies have shown that CPB, through the non-natural event of blood contacting polymeric surfaces, i.e., the surface of a polymer layer, induces a whole-body inflammatory response involving cellular and non-cellular elements of blood. First, red cells are damaged due to shear stress. This causes cell lysis, which results in a release of hemoglobin and production of membrane “ghosts,” which are membranes of red blood cells without any internal content. Further, both neutrophils and vascular endothelial cells are activated by CPB. Neutrophils adhere to endothelium and degranulate, releasing cytotoxic substances and causing small vessel obstruction. Platelets activate, degranulate, and adhere to CPB components.

Exposure of blood to plastic tubes and machine components, otherwise referred to as an extracorporeal circuit, activates the complement and contact system. A humoral inflammatory cascade begins with activation of Hagerman factor (factor XII). In the contact system, the active form of factor XII converts pre-kallikrein to kallikrein and starts the intrinsic coagulation cascade, and eventually initiates the extrinsic pathway. This leads to the formation of thrombin and drives inflammation. The thrombin will turn into fibrin, which results in clot formation. Thrombin also induces endothelial cells to produce platelet activating factor (PAF), another potent activator of neutrophils, and directly influences neutrophils to express pro-inflammatory cytokines. Such platelets can alter tissue integrity. In the complement system, following activation, several peptides are generated that help to increase the number of circulating leukocytes, promote leukocyte adhesion to vascular endothelium, and attract phagocytes to the sites of inflammation. Complement activation during surgery requiring CPB may play a particularly important role in the development of perioperative tissue injury due to the pro-inflammatory effects of the terminal complement products of C5 cleavage, C5a, and C5b-9. Such products mediate cellular damage, alteration of vascular permeability and tone, leukocyte chemotaxis, initiation of cardiac myocyte apoptosis, initiation of thrombosis, and promotion of both cellular activation and adhesion.

The cytokines often associated with release during and after CPB include TNF-a, IL-1b, IL-2, IL-6, IL-8, and IL-10. TNF-a and IL1B are elevated early following cardiac surgery, with IL-6 and IL-8 peaking later. TNF-a acts as a negative inotrope. Increased levels of pro-inflammatory cytokines have generally been associated with negative outcomes after cardiac surgery.

Pro-inflammatory cytokines and endotoxins can induce the release of nitric oxide (NO) by endothelial cells and smooth muscle cells through the inducible form of the enzyme NOS (iNOS). Effects of nitric oxide include vascular smooth muscle relaxation (leading to hypotension), myocardial depression, and lung injury events seen after cardiac surgery.

In ECMO and CPB, activated platelets conjugate both between themselves and with leukocytes. The platelet-leukocyte interaction induces leukocytes to secrete pro-inflammatory cytokines and monocytes, involving them in the inflammatory reaction. Factor XII activates the intrinsic coagulation cascade, kallikrein, bradykinin, and plasmin (through kallikrein). Endotoxin circulates in high concentrations after CPB.

In response to this chemical cascade, cellular and humoral immune function is depressed after CPB, which can lead to significant injury to the pulmonary, renal, and central nervous system pathology. For example, numbers and functions of T and B lymphocytes and killer T cells decrease after CPB. Altered T-call plasma membrane is also seen after CPB. Further, there is decreased lymphocyte response to PHA and decreased IL-2 production by lymphocytes after CPB. This impact on the immune system may lead to development of systemic inflammatory response syndrome (SIRS) in the patient. A frequent complication of SIRS is the development of organ dysfunction, including acute lung injury, shock, renal failure, and multiple organ dysfunction syndrome.

While portions of this cascade are attributed to CPB or ECMO, specifically, they all result from blood contact with incompatible non-blood contacting surfaces, such a plastic tubing. Such surfaces are present in current dialysis, CPB, and ECMO treatments, and therefore a similar chemical cascade inducing an immunological insult and response will occur during any of these treatments, or other treatments, using such surfaces that come into contact with blood that is eventually reintroduced into the patient's body.

The concept of biocompatibility has been researched with the goal of creating a material, or a treatment to existing materials, which avoids this immunological insult. For example, a heparin coating along plastic materials has been used in dialysis, CPB, and ECMO, and has helped, but not eliminated, the immunological insult and response. The underlying polymers of the plastic components of the medical devices used in these treatments are still non-compatible biomaterial, and the heparin coating reduces, but does not eliminate, contact between the blood and plastic components.

While the offered studies have focused on the immunological complication caused by CPB and ECMO, patients undergoing dialysis experience similar immunological complications due to blood contact with components of the extracorporeal circuit of hemodialysis and peritoneal dialysis systems.

Patients with chronic kidney failure usually have problems with fluid levels, as they have problems passing urine. Excess fluid in the body can cause a number of issues throughout different organs and systems of the body. An explanation is provided of just a few of the complications that a nephrologist will watch out for, and try to prevent, in patient's undergoing dialysis, and especially hemodialysis.

Hypotension, or low blood pressure, is a common occurrence during hemodialysis in which the dose and speed of the procedure can cause a too-rapid removal of fluids from the blood. By doing so, internal pressure in blood vessels will invariably drop, sometimes precipitously. This can cause symptoms such as abdominal discomfort, yawning or sighing, nausea, vomiting, muscle cramps, restlessness, anxiety, dizziness or fainting, clammy skin, and/or blurred vision. A severe drop in blood pressure also increases the risk of blood clots. If left untreated, the formation of clots may require additional surgery to repair the access point and, in some cases, lead to stroke, seizures, and heart damage. Adhering to recommended fluid restrictions can help. By limiting fluid intake, the amount of fluid being extracted during dialysis will be decreased, and any drop in blood pressure can be minimized.

Hemodialysis not only removes toxins and excess fluid from the body, but also many of the electrolytes that the body needs to function. In most cases, this won't pose a concern to patients adhering to a proper diet. However, even adherence to a proper diet may not be enough to prevent a condition known as hypokalemia in patients with diabetes or taking angiotensin-receptor blockers (ARBs). Hypokalemia is abnormally low potassium in the blood. Potassium is one of the most important electrolytes that the body uses to regulate fluid balance, muscle contractions, and nerve signals. When potassium levels drop excessively, it can affect all of these functions, causing fatigue, weakness, constipation, muscle cramping, and/or heart palpitations. If hypokalemia is extreme, defined as levels below 2.5 millimoles per liter (mmol/I), it can cause potentially serious complications including the breakdown of muscle tissue, ileus (lazy bowels), cardiac arrhythmia (irregular heart rate), respiratory failure, paralysis, and/or atrial or ventricular fibrillation. For most people, the risk of hypokalemia is low if they follow the prescribed diet and treatment plan. Even those at increased risk are unlikely to experience anything more than mild hypokalemia if they do.

Infection is an omnipresent risk in people undergoing hemodialysis. The creation of dialysis access supplies openings for bacteria and other microorganisms to possibly enter the bloodstream. If an infection were to occur, symptoms would typically include local swelling, redness, warmth, and pain, flatulence (the accumulation of pus beneath the skin), and fever and/or chills. Antibiotics are typically used to treat the infection. Heparin, a type of blood thinner, may be used to prevent blood clots and limb ischemia. Maintaining optimal hygiene and sanitary practices can significantly reduce the risk of infection. However, simply bumping or knocking the dialysis access can cause bleeding, especially if the graft or fistula is new. Bleeding increases the risk of infection, anemia, and vascular aneurysm (bulging of the arterial wall).

Fluid overload, also known as hypervolemia, occurs when the kidneys are no longer able to remove enough fluid from the body. If the dialysis machine is not calibrated correctly, hypervolemia may persist despite treatment. Symptoms of hypervolemia include headache, abdominal cramping and bloating, swelling of the feet, ankles, wrist, and face, high blood pressure, and/or weight gain. Adhering to fluid restrictions and tracking fluid intake can significantly reduce the risk of hypervolemia. If overload persists despite fluid restriction (or develops soon after hemodialysis), adjustments to the treatment plan can be made. If left untreated, hypervolemia can lead to heart problems, including congestive heart failure, cardiac arrhythmia, and cardiomegaly (enlargement of the heart).

Dialysis disequilibrium syndrome (DDS) is an uncommon neurological condition that typically affects people who have just started hemodialysis. It is believed to be the body's response to a procedure it considers abnormal, resulting in the release of inflammatory cytokines and other inflammatory chemicals that cause the brain to swell (cerebral edema). Symptoms of DDS include weakness, dizziness, nausea and vomiting, headache, muscle cramps, and/or changes in behavior or mental status.

These electrolyte and fluid imbalances are due to immunological insults resulting from non-natural events. They are usually short-lasting complications that will resolve if the body has enough time to adapt to the dialysis treatment. However, patients on dialysis do not live long-term, partially due to the increased stress such immunological insults put on a body that is already stressed by kidney failure.

Along with complications due to blood and body fluid contact with extracorporeal systems, dialysis sometimes requires additional procedures that cause further problems for patients. Namely, an arteriovenous (AV) fistula is a common procedure in dialysis patients wherein an artery and a vein are connected together via tubing. At the start of a dialysis treatment two needles are inserted into the AV fistula, one needle to remove blood from the body to the dialysis machine and the other needle to return the blood from the machine back to the body. The core issue with the AV fistula is arterial and venous tissues are structured for different fluidic pressures. By surgically combining the two systems at non-natural points, the normal pressure is thrown off (i.e., pressure is too low in the artery and too high in the vein) and complications can arise. An AV fistula can cause serious complications, such as lymphedema, infection, aneurysm, stenosis, congestive heart failure, steal syndrome, ischemic neuropathy, and thrombosis. In hemodialysis patients, the most common cause of vascular access failure is neointimal hyperplasia.

The immunological insults resulting from non-natural events, such as blood contact with foreign plastic surfaces, add further unwanted stress to bodies already stressed by kidney, heart, and/or lung disease. A primary goal of this invention is to provide an apparatus that does not put further immunological stress on a body experiencing failure in such organs and to alleviate patient pain and discomfort during treatment for such diseases. Another goal of this invention is to provide methods of implantation for such an apparatus and methods of treating patients using the apparatus.

### SUMMARY OF THE INVENTION

The present invention teaches a corporeal device for filtering and oxygenating blood, comprising: an elongated tubular housing having an elongated tubular frame and a bio-modified material layer, the tubular frame coaxially expandable and collapsible relative to a central axis extending through the tubular frame, the bio-modified material layer covering at least an entire outer surface of the tubular frame, and a cavity defined by the bio-modified material layer and the tubular frame, wherein the elongated tubular housing has an opening along a longitudinal end of the tubular housing; a first pouch connected to a first tube, the first tube extending into the cavity via the opening; and a second pouch connected to a second tube, the second tube extending into cavity via the opening, wherein the device is configured to create at least one differential across the bio-modified material layer between the blood outside the tubular housing and a differential matter held within the cavity, and wherein the bio-modified material layer is semi-permeable.

The corporeal device may further include a third pouch connected to a third tube, the third tube extending into the cavity via the opening. The third pouch defines a third pouch cavity connected to the third tube, an entire outer surface of the third pouch and third tube covered in a third pouch bio-modified material layer. The first tube may be shorter than the third tube by at least 15 mm. The second tube may be 15 mm in length.

In another embodiment, the first pouch defines a first pouch cavity fluidly connected to the first tube, an entire outer surface of the first pouch and the first tube covered in a first pouch bio-modified material layer; and wherein the second pouch defines a second pouch cavity fluidly connected to the second tube, an entire outer surface of the second pouch and the second tube covered in a second pouch biomodified material layer.

In another embodiment, the tubular frame is made from a memory-shape material. The memory-shape material can be nitinol.

The present invention also teaches a corporeal device for filtering and oxygenating blood, comprising: an elongated tubular housing, the tubular housing being semi-rigid, deformable from an operational shape, and reformable to the operational shape, the tubular housing defining an inner cavity, wherein the inner cavity has an opening along a longitudinal end of the tubular housing; a first pouch connected to a first tube, the first tube extending into the inner cavity via the opening; and a second pouch connected to a second tube, the second tube extending into the inner cavity via the opening, wherein the tubular housing has a bio-modified material layer covering at least an entire outer surface of the tubular housing, and wherein the device is configured to create at least one differential across the bio-modified layer between the blood outside of the tubular housing and a differential matter contained within the inner cavity.

For this corporeal device, the bio-modified material layer can be semi-permeable. The opening can be sealed around the first tube and the second tube. The differential matter can be a gas, which can contain oxygen. The differential matter can also be a fluid.

The inner cavity, a hollow portion of the first tube, a hollow portion of the second tube, a first pocket defined by the first pouch, and a second pocket defined by the second pouch define an inner environment of the device. The inner environment of the device is sealed from an outer environment and is only permeable across the bio-modified material layer of the tubular housing.

The corporeal device can further include a third pouch connected to a third tube, the third tube extending into the inner cavity via the opening. The third pouch defines a third pocket connected a hollow portion of the third tube, both the third pocket and the hollow portion of the third tube open to the inner cavity. An entire outer surface of the third pouch and the third tube are covered with a third pouch bio-modified material layer.

An entire outer surface of the first pouch and the first tube are covered with a first pouch bio-modified material layer, and an entire outer surface of the second pouch and the second tube are covered with a second pouch bio-modified material layer.

The corporeal device can be used to perform dialysis, cardio-pulmonary bypass, or extra-corporeal membrane oxygenation

The corporeal device can be fully implanted with a human body. The elongated tubular housing is positioned within a blood vessel of the human body. The first tube and the second tube bisect a wall of the blood vessel. The first pouch and the second pouch are positioned beneath a layer of skin of the human body. The wall of the blood vessel is fluidly sealed around the first tube and the second tube.

The present invention also teaches an extracorporeal device for filtering and oxygenating blood, comprising: a pump apparatus having a pump housing defining an inner chamber, the pump housing having two opposing longitudinal ends, each of the two opposing longitudinal ends having a valve movable between an open position and a closed position, a flexible tube of bio-modified material extending between and sealed around each said valve, such that a fluid is flowable through an openable and closable channel defined by each said valve and the flexible tube of bio-modified material, and an air pump operably secured to the housing via an air connection opening; a differential apparatus having a differential housing and two opposing longitudinal ends, the differential housing and two opposing longitudinal ends defining a differential chamber, an entire inner surface of the differential chamber covered in a chamber layer of bio-modified material, a differential member longitudinally extending within the differential chamber, an outer layer of the differential member covered in a member layer of bio-modified material, the member layer being semi-permeable, the differential member defining an inner differential cavity, a first tube fluidly connected to the inner differential cavity and to a first container, the first container and at least a partial length of the first tube positioned externally to the differential housing, and a second tube fluidly connected to the inner differential cavity and to a second container, the second container and at least a partial length of the second tube positioned externally to the differential housing; an extraction member insertable into a blood vessel and fluidly connected to the differential apparatus; and an insertion member insertable into a blood vessel and fluidly connected to the pump apparatus; wherein the differential apparatus is fluidly connected to the pump apparatus.

The inner chamber of the pump apparatus includes an air chamber and a fluid chamber, the fluid chamber defined by the flexible tube and each said valve, and the air chamber defined between the pump housing and the flexible tube. The extracorporeal can further include an opening along the pump housing, wherein the air pump is operably connected to the opening. The air pump is configured to add and remove air from the air chamber to increase and decrease pressure applied to the fluid chamber.

A tube connects the air pump to the opening, and the tube may threadingly engage the opening.

The pump apparatus runs between a diastole phase and a systole phase to move fluid through the extracorporeal device.

The present invention also teaches a differential apparatus for filtering or oxygenating blood, comprising: a housing defining an interior cavity and having two opposing longitudinal ends, the housing having an opening along each longitudinal end of the two opposing longitudinal ends that is fluidly connected to the interior cavity; a pair of elongated members positioned within the interior cavity and spaced apart; a bio-modified layer wrapped around the pair of elongated members, and one or more pairs of access openings in the housing, wherein the bio-modified layer and the pair of elongated members define an inner chamber and an outer chamber within the interior cavity, wherein each said opening is fluidly connected to the inner chamber, and wherein the one or more pairs of access openings are fluidly connected to the outer chamber.

The present invention also teaches a method of corporeally filtering or oxygenating blood, comprising: inserting an implantable filtration device within a blood vessel of a patient, wherein the implantable filtration device includes an elongated tubular housing, the tubular housing being semi-rigid, deformable from an operational shape, and reformable to the operational shape, the tubular housing defining an inner cavity, wherein the inner cavity has an opening along a longitudinal end of the tubular housing; a first pouch connected to a first tube, the first tube extending into the inner cavity via the opening; and a second pouch connected to a second tube, the second tube extending into the inner cavity via the opening, wherein the tubular housing has a bio-modified material layer covering at least an entire outer surface of the tubular housing, and wherein the device is configured to create at least one differential across the bio-modified layer between the blood outside of the tubular housing and a differential matter contained within the inner cavity; connecting an introduction line to the first pouch, such that extracorporeal differential matter moves from the introduction line, into the first pouch, through the first tube and into the inner cavity of the elongated tubular housing; and connecting a removal line to the second pouch, such that waste matter is movable from the inner cavity of the elongated tubular housing, through the second tube, into the second pouch, and out of the filtration device through the removal line.

The method may further include filtering the blood corporeally by removing waste from the blood via the least one differential across the bio-modified layer through the extracorporeal differential matter within the inner cavity. The extracorporeal differential matter is a fluid designed to filter blood of a human experiencing kidney failure.

The method may further include oxygenating the blood corporeally via the least one differential across the bio-modified layer through the extracorporeal differential matter within the inner cavity. The extracorporeal differential matter is a gas having oxygen.

The first pouch and the second pouch are positioned under skin of the patient, and the introduction line and the removal line are inserted through the skin to connect to the first pouch and the second pouch. The first pouch, the second pouch, the first tube and the second tube are all covered in a secondary bio-modified layer. The bio-modified layer covering the tubular housing and the secondary bio-modified layer do not cause an immunological response within a body of the patient. Matter can only enter or leave the filtration device due to the at least one differential along the bio-modified layer covering the tubular housing.

The present invention also teaches a method of extra-corporeally filtering or oxygenating blood, comprising: attaching an extracorporeal filtration device to a patient, wherein the extracorporeal filtration device includes a pump apparatus having a pump housing defining an inner chamber, the pump housing having two opposing longitudinal ends, each of the two opposing longitudinal ends having a valve movable between an open position and a closed position, a flexible tube of bio-modified material extending between and sealed around each said valve, such that fluid flows through an openable and closable channel defined by each said valve and the flexible tube of bio-modified material, and an air pump operably secured to the pump housing via an air connection opening, a differential apparatus having a differential housing and two opposing longitudinal ends, the differential housing and two opposing longitudinal ends defining a differential chamber, an entire inner surface of the differential chamber covered in a chamber layer of bio-modified material, a differential member longitudinally extending within the differential chamber, an outer layer of the differential member covered in a member layer of bio-modified material, the member layer being semi-permeable, the differential member defining an inner differential cavity, a first tube fluidly connected to the inner differential cavity and to a first container, the first container and at least a partial length of the first tube positioned externally to the differential housing, and a second tube fluidly connected to the inner differential cavity and to a second container, the second container and at least a partial length of the second tube positioned externally to the differential housing, an extraction member insertable into a blood vessel and fluidly connected to the differential apparatus, and an insertion member insertable into a blood vessel and fluidly connected to the pump apparatus, wherein the pump apparatus is fluidly connected to the differential apparatus, extracting blood from a first blood vessel through the extraction member inserted into the first blood vessel; pumping blood into and out of the extracorporeal filtration device through alternating diastole and systole phases of the pump apparatus achieved through adding and removing air in the inner chamber via the air pump; creating at least one differential across the chamber layer of bio-modified material between the blood within the differential chamber and differential matter contained within the differential member; adding and/or removing matter from the blood while in the differential chamber, returning the blood to a second blood vessel via the insertion member inserted into the second blood vessel.

The differential matter has oxygen, and the blood is oxygenated while in the differential chamber, or the differential matter contains a fluid designed to remove waste from the blood, and the blood is filtered while in the differential chamber.

The present invention also teaches a method of filtering or oxygenating blood using an artery-to-artery connecting, comprising: surgically connecting a superior artery to an inferior artery with a tubular graft within a patient; connecting the insertion member and the extraction member of the previously described extracorporeal device to the graft; and maintaining similar internal fluid pressure within the superior artery and the interior artery during the filtering or oxygenating of the blood using the extracorporeal device.

A diffusion device is also taught, having a housing defining an interior cavity and having two opposing longitudinal ends, the housing having an opening along each longitudinal end of the two opposing longitudinal ends that is fluidly connected to the interior cavity; a tissue frame positioned within and extending across the interior cavity to define two or more fluid chambers within the interior cavity, each fluid chamber of the two or more fluid chambers separated from another fluid chamber of the two or more fluid chambers by a semi-permeable membrane layer of the tissue frame; wherein each said opening is fluidly connected a primary chamber of the two or more fluid chambers.

In the diffusion device, the tissue frame may further include a frame member forming a loop and a tissue layer attached to at least one side of the frame member, the tissue layer forming the semi-permeable membrane layer. The housing may have two housing halves securable together to form the inner cavity. Also in the diffusion device, where the at least one side of the housing has an input port and an output port, the input port and the output port fluidly connecting an external environment to a corresponding fluid chamber of the two or more fluid chambers.

The embodiments of the invention will be better understood with reference to the drawings and the following brief description of the several views of the drawings.

### BRIEF DESCRIPTION OF THE SEVERAL VIEWS OF THE DRAWINGS

FIG. 1A is a cross-sectional view along a length of an embodiment of a tubular housing;

FIG. 1B is a cross-sectional view of the tubular housing of FIG. 1A along a plane 1B-1B in a direction of arrows provided in FIG. 1A;

FIG. 2A is a cross-sectional view along a length of a corporeal device for filtration and oxygenation, which includes the tubular housing of FIG. 1A, having three pouches and corresponding tubes;

FIG. 2B is a cross-sectional view of the corporeal device of FIG. 2A along a plane 2B-2B;

FIG. 2C is a side view of tubes of the corporeal device of FIG. 2A with the tubular housing of FIG. 1A and pouches of FIG. 2A shown in dotted lines to indicate they have been removed to view the tubes housed therein;

FIG. 3 is a side view of alternative embodiment of the corporeal device of FIG. 2A-2C having three tubes ending at different locations along a length of the tubular housing of the corporeal device;

FIG. 4 is an expanded view along an end of a third tube according to an embodiment of the corporeal device;

FIG. 5 is a diagram along a partial length of corporeal device embodiment implanted in a blood vessel, the three tubes of the corporeal device shown at different lengths;

FIG. 6A is an illustration of the corporeal device implanted in a blood vessel along a cross-section taken along a width of the corporeal device and blood vessel, the illustration showing representations of a differential across a bio-modified layer;

FIG. 6B is an illustration along a cross-section of the corporeal device and the blood vessel, as shown in FIG. 6A, with a portion of a group of symbols representing chemical constituents permeating across the bio-modified layer.

FIG. 7 is a side view of another embodiment of the corporeal device, which includes the tubular housing of FIG. 1A, having two pouches and corresponding tubes;

FIG. 8 is an illustration of a patient laying on a table, the corporeal device of FIG. 2A operably inserted into the patient's body and operably connected to external fluid bags via two of three pouches for performing dialysis;

FIG. 9A is a cross section of the pouches of the corporeal device operably inserted into the patient's body, as shown in FIG. 5A;

FIG. 9B is a side view of a second pouch of the corporeal device of FIG. 9A, the first pouch receiving an inserted out-line for removing fluid from the body;

FIG. 9C is a side view of a first pouch of the corporeal device of FIG. 9A, the second pouch receiving an inserted in-line for returning fluid to the body;

FIG. 9D is a side view of a third pouch of the corporeal device of FIG. 9A, the third pouch receiving an insertable element;

FIG. 10A is a perspective view of another embodiment of the tubular housing;

FIG. 10B is a cross-sectional view of the tubular housing of FIG. 10A along the cross-section C-C shown in FIG. 10A;

FIG. 10C is a side view of the tubular housing of FIG. 7A;

FIG. 10D is a cross-sectional view of an alternative embodiment of the tubular housing shown in FIG. 10A;

FIG. 11 is an illustration of a cardiovascular system of a human body with two corporeal device systems operably inserted, one corporeal device inserted within a vein and another corporeal device inserted within an artery;

FIG. 12 is a perspective view of an embodiment of a para-corporeal device;

FIG. 13 is a perspective view of a bio-pump of the para-corporeal device of FIG. 12;

FIG. 14A is an illustration of the bio-pump of FIG. 12 during a diastole phase;

FIG. 14B is an illustration of the bio-pump of FIG. 12 during a systole phase;

FIG. 15 is an alternative embodiment of the para-corporeal filtration device;

FIG. 16 is an illustration of the para-corporeal device of FIG. 12 operably secured to a patient's body;

FIG. 17 is an illustration of a graft implanted in a body for an artery-to-artery connection of the para-corporeal device;

FIG. 18A is a perspective view of an alternative embodiment of a differential device for para-corporeal filtration or oxygenation;

FIG. 18B is a cross-sectional view along plane 18B-18B of the differential device of FIG. 18A;

FIG. 18C is a cross-sectional view along plane 18C-18C of the differential device of FIG. 18B;

FIG. 18D is a cross-sectional view along plane 18D-18D of the differential device of FIG. 18B;

FIG. 18E is a cross-sectional view along plane 18D-18D of an alternative embodiment of the differential device of FIG. 18B;

FIG. 19 is a top plan view of a half of an alternative embodiment of a differential device;

FIG. 20 is a top plan view of a tissue frame embodiment;

FIG. 21 is a perspective view of another tissue frame embodiment;

FIG. 22 is a top plan view of the tissue frame embodiment of FIG. 20 covered on one side with a layer of tissue;

FIG. 23 is a top plan view of the tissue frame embodiment of FIG. 20 encased by tissue;

FIG. 24 is a perspective view of a differential device housing the tissue frame embodiment of FIG. 20;

FIG. 25 is an exploded view of the differential device of FIG. 24, also showing the tissue frame embodiment of FIG. 20;

FIG. 26 is a plan view along an inside of two halves of a housing of the differential device of FIG. 24, with the encased tissue frame embodiment of FIG. 23 positioned within one of the two halves of the housing;

FIG. 27A is a perspective view of the differential device of FIG. 26;

FIG. 27B is a top plan view of the differential device of FIG. 27A;

FIG. 27C is side plan view of the differential device of FIG. 27A;

FIG. 27D is a front plan view of the differential device of FIG. 27A;

FIG. 27E is a back plan view of the differential device of FIG. 27A;

FIG. 27F is a bottom plan view of the differential device of FIG. 27A;

FIG. 28A is a cross-sectional view of the differential device of FIG. 27A along axis 28A;

FIG. 28B is a cross-sectional view of the differential device of FIG. 27A along axis 28B;

FIG. 29A is a perspective view of a differential device embodiment;

FIG. 29B is a top plan view of the differential device embodiment of FIG. 29A;

FIG. 29C is side plan view of the differential device embodiment of FIG. 29A;

FIG. 29D is a front plan view of the differential device embodiment of FIG. 29A;

FIG. 29E is a back plan view of the differential device embodiment of FIG. 29A;

FIG. 29F is a bottom plan view of the differential device embodiment of FIG. 29A;

FIG. 30A is a side plan view of a differential device embodiment similar to the differential device embodiment of FIG. 29A;

FIG. 30B is a cross-sectional view of the differential device of FIG. 30A along axis 30B;

FIG. 30C is a side plan view of a differential device embodiment of FIG. 30A; and

FIG. 30D is a cross-sectional view of the differential device of FIG. 30A along axis 30D.

### DETAILED DESCRIPTION OF THE INVENTION

As described herein, “bio-modified,” in terms of a bio-modified tissue or a bio-modified layer, means a tissue, material, or layer that is repurposed and/or treated organic tissue, such as treated animal pericardium tissue or replicated organic tissue, such as cloned animal or human tissue. Preferably, such a tissue, material, or layer was naturally, or is replicated as, a blood-contacting surface. One example of a bio-modified tissue is porcine pulmonary valve and bovine pericardium affixed with glutaraldehyde and then detoxified with a proprietary process called No-React®, as described in Ghiselli S, Carro C, Uricchio N, Annoni G, Marianeschi SM. Mid- to long-term follow-up of pulmonary valve replacement with BioIntegral injectable valve. Eur J Cardiothorac Surg 2020; doi:10.1093/ejcts/ezaa337. A bio-modified tissue, material, or layer does not create a non-natural event when in contact with human blood and does not induce a resulting immunological insult in a patient's body.

As described herein, “corporeal,” in relation to described embodiments of the invention and use of the same in filtration or oxygenation, means filtration or oxygenation that occurs within a living body, such as a human body. In terms of blood filtration and/or oxygenation, corporeal means that blood is not removed from inside the body and either waste is removed from the blood or oxygen is introduced into the blood as it moves along normal blood vessel pathways throughout the body.

As described herein, “extra-corporeal,” in relation to described embodiments of the invention and use of the same in filtration or oxygenation, means filtration or oxygenation that occurs outside of a living body, such as a human body. In terms of blood filtration and/or oxygenation, extra-corporeal means that blood is first removed from inside the body, waste is removed from the blood or oxygen is introduced into the blood while it is outside of the body, and the filtered and/or oxygenated blood is then returned to the body to then travel along normal blood vessel pathways throughout the body.

As described herein, “polymeric surfaces,” means foreign, non-natural, or synthetic surfaces that are reactive with bodily fluids or tissues to incite a chemical cascade that results in an immunological response inside a human body. The reaction between bodily fluids or tissues and such surfaces may occur outside of the body and propagate the immunological response once such fluids or tissues are reintroduced (extracorporeal), or the reaction may occur within the body due to such surfaces being inserted or implanted in the body (corporeal).

As described herein, “differential matter,” means solids, liquids, and/or gases introduced into one or more embodiments of the present invention to create one or more differentials between the solids, liquids, and/or gases and blood in a human body across a semi-permeable membrane.

As described herein, “treatment fluid,” is a differential matter and a fluid introduced into a device of the present invention to create one or more differentials across a semi-permeable membrane with the purpose of removing waste from blood in a human body.

As described herein, “waste fluid,” means fluid containing waste that is removed from the device and from the patient's body.

As described herein, “cavity fluid,” means the fluid contained within an internal cavity of a device at any given point of time, such fluid being separated across a semi-permeable membrane from a fluid that is targeted for filtration and/or oxygenation, which is preferably blood. The cavity fluid may be a mix of treatment fluid, waste fluid, medication, compounds, molecules, fluids, and other differential matter.

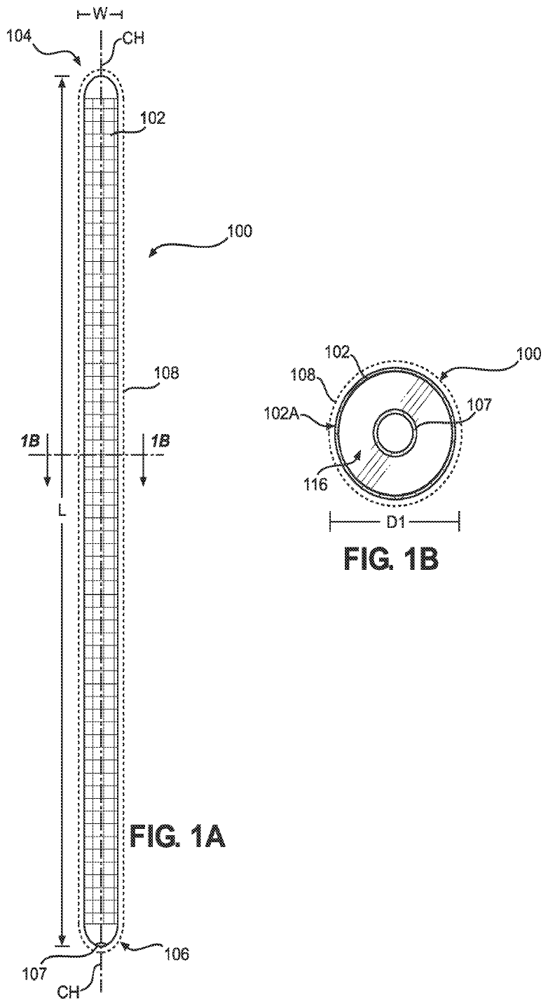

FIG. 1A illustrates a cross-sectional view along a length L of an elongated tubular housing 100 having a tubular frame 102, otherwise referred to as a stent frame. The stent frame 102 is coaxially collapsible and expandable about a central axis CH longitudinally extending through a center of the stent frame. The stent frame 102 has two opposing longitudinal ends 104 and 106. The tubular housing further includes a bio-modified layer 108 covering at least an entire outer surface 102a of the stent frame 102. A length L of the tubular housing may vary between 152.4-355.6 mm (millimeters), or 6-14″ (inches). The stent frame 102 may be made from a metal or metal alloy, preferably a metal or metal alloy that has a memory shape or can be deformed from a given shape and then automatically return to the given shape, such as nitinol. The stent frame 102 may be made from material that is not a metal, such as plastics or other polymers, as the stent frame does not come into contact with blood or bodily fluids. The stent frame 102 is preferably made from a material that is deformable, but returns to a given shape after being deformed, such as being collapsed to fit through an opening or structure during implantation.

A cross-section along a width W of the tubular housing 100 is shown along plane 1B-1B in FIG. 1B. Along this cross-section, the bio-modified layer 108 is shown covering the outer surface 102A of the stent frame 102 to form an inner cavity 116 within the tubular housing 100. The tubular housing is closed at the end 104 by the bio-modified layer 108. However, there is an opening 107 along the end 106 to allow one or more structures to be inserted into the cavity 116 via the opening. In an operably expanded diameter, or the diameter after implantation and during use for filtration and/or oxygenation, the tubular housing 100 preferably has a diameter D₁ between 6-9 mm. A preferable implantation site for the tubular housing is in an aorta. The aorta in an average adult has a diameter of 18-36 mm, which provides ample room for blood to flow around the tubular housing 100 should it be implanted in the aorta. Implantation in other blood vessels is contemplated, including in either arteries or in veins of sufficient size to allow sufficient blood flow around the tubular housing 100. The diameter D₁ of the tubular housing 100 may likewise be smaller or larger to accommodate smaller larger blood vessels.

The bio-modified layer 108 is shown in broken lines in FIGS. 1A-1B to denote its semi-permeable qualities. However, the bio-modified layer 108 fully covers the outer surface 102A, other than the opening 107, of the stent frame 102 to help define the inner cavity 116 and block blood flow into the inner cavity across the stent frame.

Other embodiments of the tubular housing 100 without the tubular frame 102 are possible. In such embodiments, the tubular housing 100 would be semi-rigid, capable of deforming and reforming to an operable shape, and having an outer bio-modified layer 108 such that blood only contacts the bio-modified outer layer upon and after implantation of the tubular housing 100 in a blood vessel of a human body. The bio-modified layer 108 may be a separate sheet applied over an outer surface of the tubular housing 100 or stent frame 102, or it may be integrated with the tubular housing such that the tubular housing is made of bio-modified material.

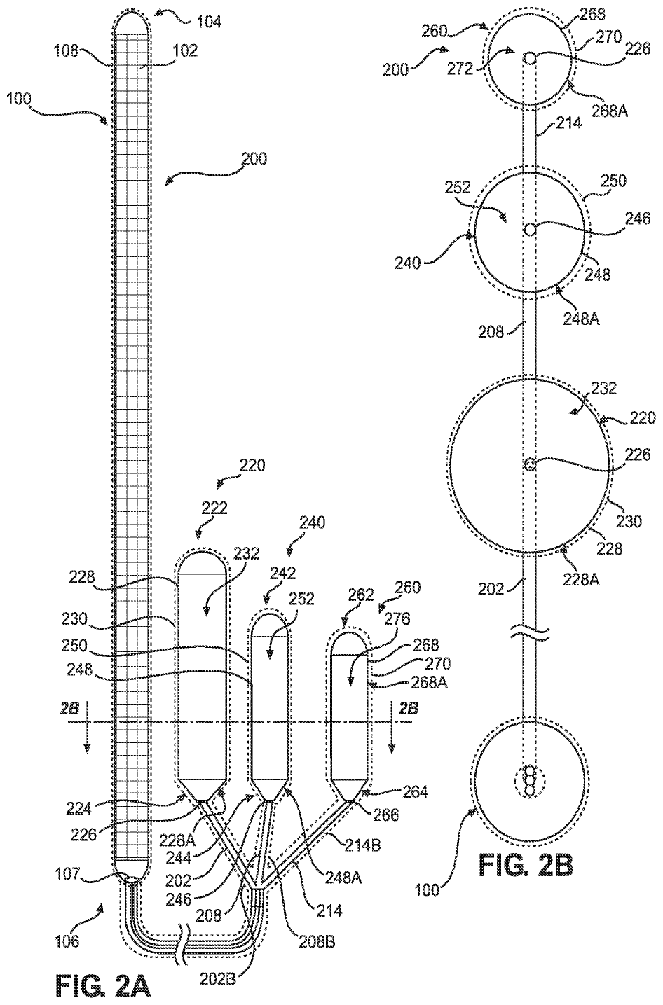

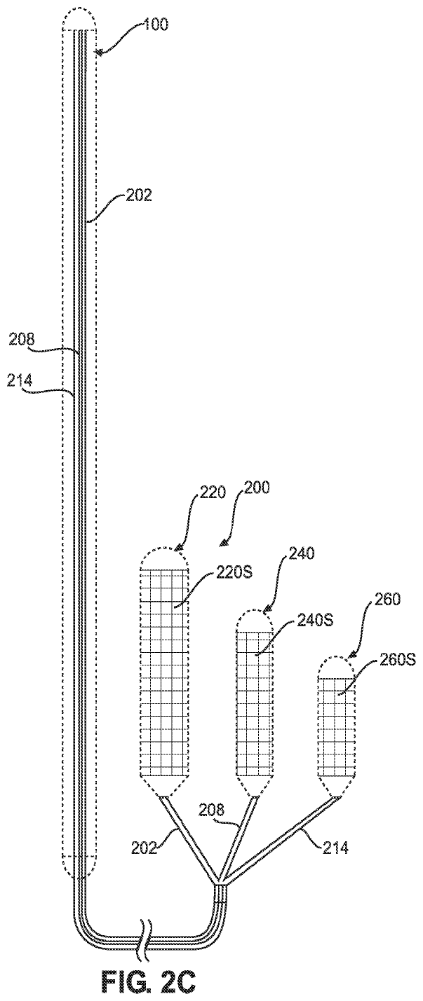

FIGS. 2A-2C illustrates an embodiment of a corporeal device 200, which includes the tubular housing 100 and three pouches 220, 240, and 260. The three pouches 220, 240, and 260 are secured to the tubular housing 100 by three respective tubes 202, 208, and 214. The three tubes 202, 208, and 214 extend through the opening 107 and into the inner cavity 116 of the tubular housing. The opening 107 is sealed around the three tubes 202, 208, and 214. Fluid can travel between the inner cavity 116 and the three pouches 220, 240, and 260 via the three tubes 202, 208, and 214. However, fluid in the inner cavity 116 cannot pass into or freely mix with fluid in an external environment. Also, fluid flowing from one or more of the three pouches 220, 240, and 260 via one or more of the three tubes 202, 208, and 214 to the inner cavity 116 cannot freely mix or escape to the external environment. The bio-modified layer 108 is semi-permeable, and differential matter may be introduced into the inner cavity 116 via one or more pouches 220, 240, and 260 and tubes 202, 208, and 214 to create osmotic or chemical differentials between an internal environment in the inner cavity 116 and the external environment across the bio-modified layer of the tubular housing 100. In response to such differentials, the bio-modified layer 108 is configured to allow differential matter to pass across the bio-modified layer between the external environment and the inner cavity 116.

The first pouch 220 in this embodiment is elongated and has two opposing longitudinal ends 222 and 224. The first pouch 220 may also have a first pouch housing 228 that provides shape to the first pouch and defines an inner cavity 232 of the first pouch. A bio-modified layer 230 covers an outer surface 228A of the first pouch housing. The inner cavity 232 is fluidly connected to the first tube 202 through an opening 226 in the first pouch housing 228 found along the end 224. The first pouch 220 may be made of a flexible material, such as silicone or similar material. The first pouch 220 may also include a frame attached to or secured within the housing 228 to provide added support. The frame may be structured similarly to the stent frame 102. In either embodiment, the bio-modified layer 230 covers the entire outer surface 228A of the first pouch 220. While the bio-modified layer 230 may be semi-permeable, the housing 228 is not. Therefore, the housing 228 must be punctured by a needle or similar device to introduce external matter, typically either fluids or air, into the cavity 232.

The second pouch 240 in this embodiment is elongated and has two opposing longitudinal ends 242 and 244. The second pouch 240 may also have a second pouch housing 248 that provides shape to the second pouch and defines an inner cavity 252 of the second pouch. A bio-modified layer 250 covers an outer surface 248A of the second pouch housing. The inner cavity 252 is fluidly connected to the second tube 208 through an opening 246 in the second pouch housing 248 located along the end 244. The second pouch 240 may be made of a flexible material, such as silicone or similar material. The second pouch 240 may also include a frame attached to or secured within the housing 248 to provide additional support. The frame may be structured similarly to the stent frame 102. In either embodiment, the bio-modified layer 250 covers the entire outer surface 248A of the second pouch 240. While the bio-modified layer 250 may be semi-permeable, the housing 248 is not. Therefore, the housing 248 must be punctured by a needle or similar device to introduce external matter, typically either fluids or air, into the cavity 252.

The third pouch 260 in this embodiment is elongated and has two opposing longitudinal ends 262 and 264. The third pouch 260 may also have a third pouch housing 268 that provides shape to the third pouch and defines an inner cavity 262 of the third pouch. A bio-modified layer 270 covers an outer surface 268A of the third pouch housing. The inner cavity 272 is fluidly connected to the third tube 214 through an opening 266 in the third pouch housing 268 found along the end 264. The third pouch 260 may be made of a flexible material, such as silicone or similar material. The third pouch 260 may also include a frame attached to or secured within the housing 268 to provide added support. The frame may be structured similarly to the stent frame 102. In either embodiment, the bio-modified layer 270 covers the entire outer surface 268A of the second pouch 260. While the bio-modified layer 270 may be semi-permeable, the housing 268 is not. Therefore, the housing 268 must be punctured by a needle or similar device to introduce external matter, typically either fluids or air, into the cavity 272.

Bio-modified layers 230, 250, and 270 are shown in broken lines to indicate that the outer surfaces 228A, 248A, and 268A are covered by the bio-modified layers, which are either separate layers extended over the housings 228, 248, and 268 or are applied directly to the outer surfaces.

The tubes 202, 208, and 214 are preferably made from a durable, yet flexible material. For example, the tubes 202, 208, and 214 may be made from a silicone steel alloy. Other materials known in the art and safe for medical uses, including implantation in a human body, for use as tubes for fluid or gas transport are also acceptable. Each of the tubes 202, 208, and 214 is covered by a bio-modified layer 202B, 208B, and 214B, respectively, along any respective length of the tubes that could be potentially contacted by a bodily surface. This includes any part of any tube 202, 208, or 214 that extends outside of the tubular housing 100 or the three pouches 220, 240, or 260. Each bio-modified layer 202B, 208B, and 214B is shown in dotted lines to for illustrative purposes only.

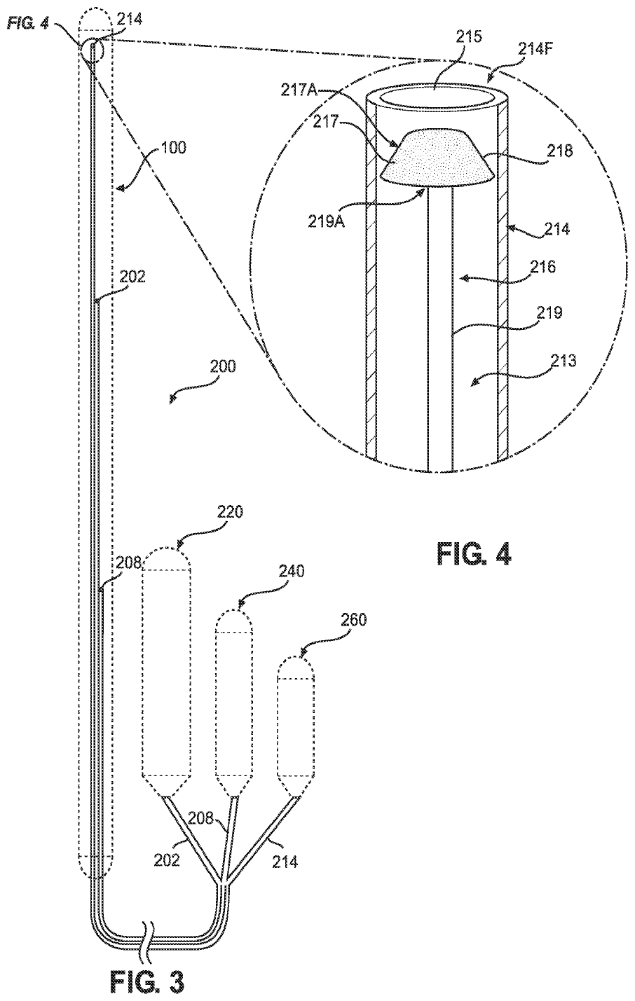

The tubes 202, 208, and 214 may extend into and end at a same location within the tubular housing 100, as shown in FIG. 2C, or may end at different locations within the tubular housing, as shown in FIG. 3. In the embodiment of the corporeal device shown in FIG. 3, the second tube 208 extends no more than 15 mm into the inner cavity 116 from the opening 107. The first tube 202 extends farther than the second tube 208, while the third tube 214 extends at least 15 mm beyond the first tube. Other orientations, relative lengths, and overall lengths of the tubes 202, 208, and 214 are permissible and are contemplated, as long as the tubes all end within the inner cavity 116. Only the tubes 202, 208, and 214 are shown in FIGS. 2C and 3. Representations of the tubular housing 100 and pouches 220, 240, and 260 are provided in dotted lines to reference where such structures would be located relative to the tubes 202, 208, and 214 in the full device 200.

Further, as shown in FIG. 2C, the pouches 220, 240, and 260 may optionally include a frame 220S, 240S, and 260S, respectively. These frames are similar to the frame 102 in the tubular housing 100 and can provide additional support to provide the pouches shape when inserted within a patient's body.

In embodiments of the corporeal device 200 having a third tube 214, an occlusion device 216 may be introduced through the third pouch 214 and into the third tube, as shown in FIG. 4. The occlusion device 216 includes a wire 219 which is thin enough to travel through a cylindrical cavity 213 of the third tube 214, and an occlusion member 217 located at an end 219a of the wire. The occlusion member 217 is shaped to move through the cylindrical cavity 213 and to occlude either the cylindrical cavity or an opening 215 along a free end 214F of the third tube 214 to prevent fluid flow past the occlusion member into or out of the third tube. The occlusion member 217 may have a heparin coating 218 along an outer surface 217A. Alternatively, a dose of heparin may be administered through the third tube 214 each time the occlusion device 216 is inserted into the third tube.

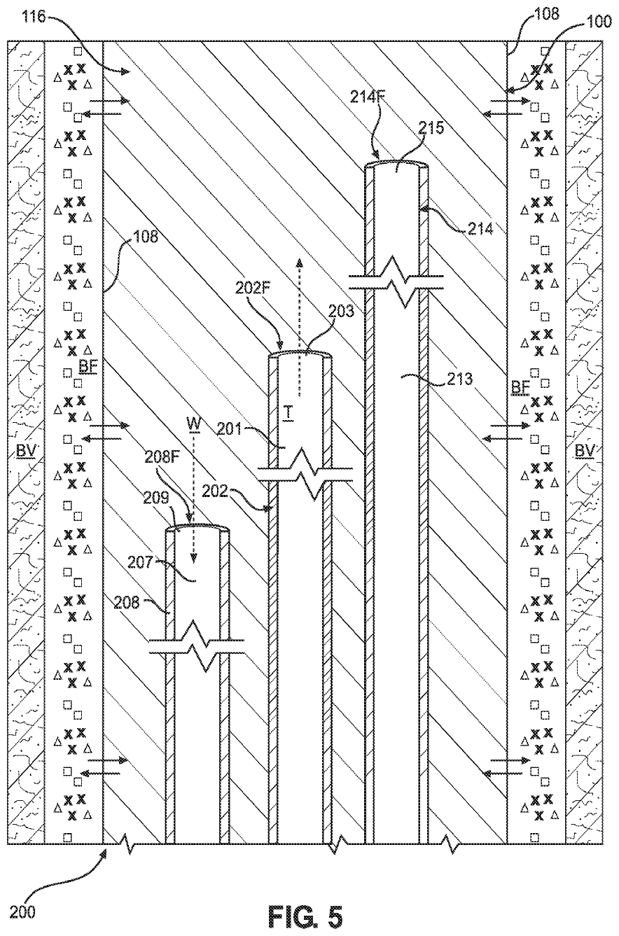

FIG. 5 shows the tubes 202, 208, and 214 of corporeal device 200, as positioned with the device is inserted in a blood vessel BV. Preferably, the device 200 is positioned such that a bodily fluid BF to be filtered or oxygenated, typically blood, completely surrounds the tubular housing 100. In the inner cavity 116, the first tube 202 introduces a treatment fluid or oxygenated air, together referred to as a treatment T, from the first pouch 220 into the inner cavity through a cylindrical cavity 201 of the first tube and out through an opening 203 along a free end 202F. Waste fluid or deoxygenated air, together referred to as waste W, is removed from the inner cavity 116 via an opening 209 along a free end 208F of the second tube 208, through a cylindrical cavity 207 in the second tube, to the second pouch 240. The third tube 214 may be used to introduce medication that is permeable through the bio-modified layer 108 into the patient's blood, or other bodily fluid BF. Additionally, the third tube 214 may be used to perform blood analysis or to measure blood pressure. When not in use, the third tube 214 may be closed via the occlusion member 216 shown in FIG. 4.

Preferably, the first pouch 220 is used to introduce a treatment T, either fluid for extracting waste from bodily fluids or oxygenate air, into the inner cavity 116 of the tubular housing 100 via the first tube 202. The composition of the treatment T may vary depending on whether blood filtration or oxygenation is the primary treatment goal, the specific treatment plan of the patient, and the waste targeted for removal from the patient's blood, or other bodily fluid. For example, the treatment T may be a fluid containing salt, sugar, etc. to create a chemical or osmotic differential between the bodily fluid BF and the cavity fluid CF. In other uses, the treatment T may be oxygenated air.

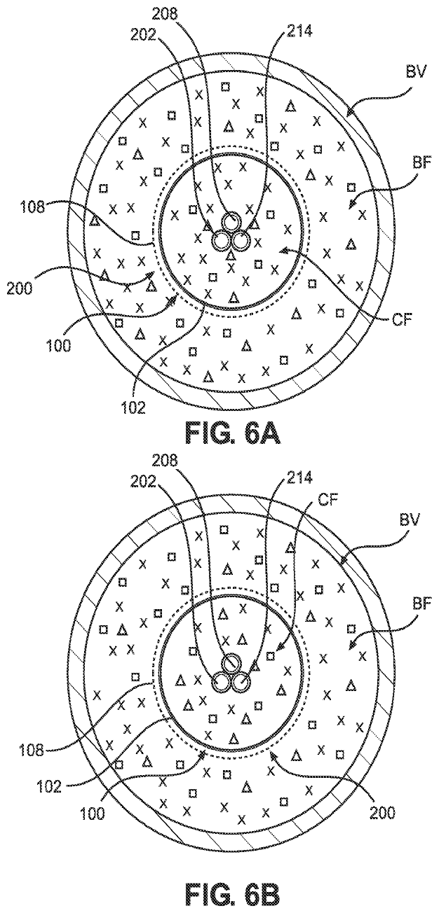

FIGS. 6A and 6B show a cross-sectional view along the corporeal device 200 and the blood vessel BV in which the corporeal device is implanted. For illustration purposes, components of the bodily fluid BF are represented as various symbols (Xs, squares, and triangles).

The treatment T, a treatment fluid for the purposes of this example, is introduced into the device 200 via the first pouch 220 and added to the cavity fluid CF through the first tube 202. The treatment fluid has differential matter used to create a differential between the bodily fluid BF, i.e., blood, and the cavity fluid CF across the semi-permeable bio-modified layer 108. For example, in FIG. 6A the cavity fluid includes Xs, squares, and triangles which are representative of possible differential matter. The blood, or bodily fluid BF, across the bio-modified layer 108 contains Xs, squares, and triangles. There is a smaller concentration of triangles inside the inner cavity 116 compared to in the blood. This creates a chemical differential that promotes movement of the triangle molecules across the semi-permeable membrane 108 and into the inner cavity 108, as shown in FIG. 6B. The triangle molecules enter the cavity fluid CF and can then be removed via the waste W removed through the second tube 208. The bio-modified layer 108 prevents certain structures and bodily fluid components, such as blood cells and other larger structures, from permeating into the inner cavity 116. Through this selective permeation, the blood is filtered to remove waste, such as excess salt, excess fluid, toxins, and similar waste material. The treatment fluid can be composed to generally reestablish homeostasis within bodily fluid, such as to lower any excess waste to a desired threshold or may be composed to target removal of a specific waste. Additionally, the treatment T can be targeted to replenish or add material to the blood. During oxygenation, oxygen is commonly added to the blood. However, other permeable materials, such as medications, may be introduced to bodily fluids BF through permeation across the modified bio-layer 108.

Once waste W has permeated into the inner cavity 116 and the cavity fluid CF, the waste fluid WF is removed from the inner cavity via the second tube 208 to the second pouch 240. The waste fluid WF is removable from the second pouch 240 via an insertable fluid-removal line, which may include a needle inserted through a layer of skin and into the second pouch or a needle directed inserted in the second pouch. In either scenario, the needle is connected to a fluid line to remove the waste fluid WF to an extracorporeal fluid container for proper, safe disposal of the waste fluid.

Preferably, the third pouch 260 is blocked when not in use, as shown in FIG. 4. When needed, the occlusion device can be removed to allow medication to be inserted into the third pouch 260 via insertion of a needle or syringe. Such medication can travel through the third tube 214 and into the inner cavity 116, mix with the cavity fluid CF and permeate across the bio-modified layer 108 into the blood or other bodily fluid of the patient. The third pouch also allows for blood testing and blood pressure measurement.

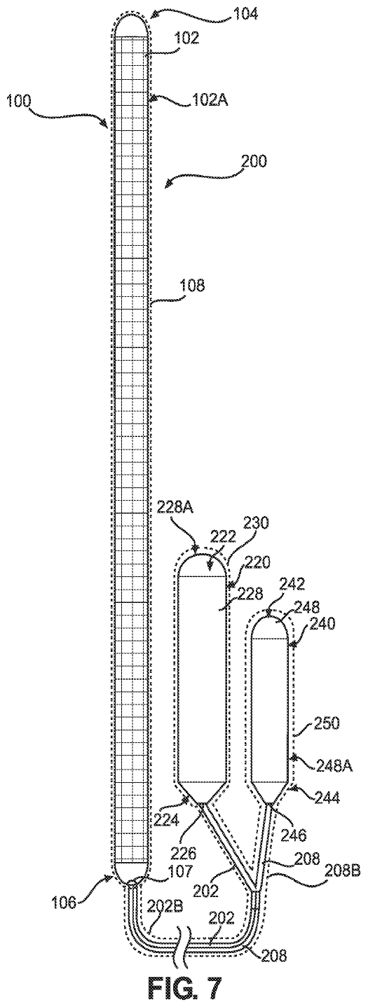

FIG. 7 illustrates an alternative embodiment of the corporeal device 200. In this embodiment, there is only a first pouch 220 and a second pouch 240, fluidly connected to the inner cavity 116 of the tubular housing 100 via a first tube 202 and a second tube 208. There is no third pouch or third tube in the device 200 of FIG. 7. It is otherwise similar to the device 200 embodiment of FIG. 2A, including that the first tube 202 and second tube 208 may terminate at the same or different length within the inner cavity 116. The device 200 embodiment of FIG. 7 is best suited for a patient performing his or her own dialysis treatment at home, whereas the embodiment of FIG. 2A is suited for supervised dialysis treatment where a physician or licensed medical professional can properly administer medication and perform tests via the third pouch 260 and third tube 214. As with the embodiment of FIG. 2A, the outer surfaces 102A, 228A, and 248A and tubes 202 and 208 are covered by a bio-modified layer 108, 202B, 208B such that blood only contacts such bio-modified layers to prevent non-natural events.

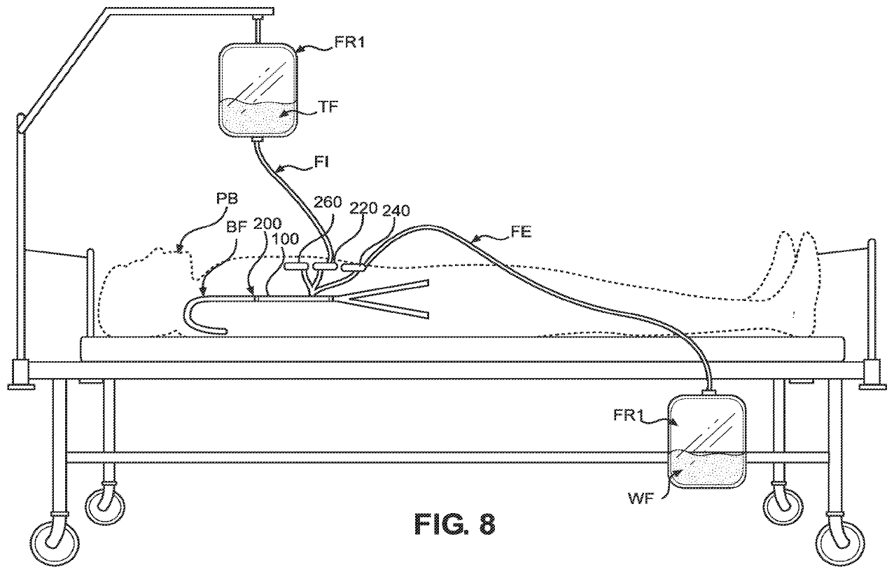

FIG. 8 shows the corporeal device 200 having three pouches 220, 240, and 260, similar or identical the embodiment of FIG. 2A, implanted into a body PB of a patient undergoing treatment, specifically filtration treatment in this representation. The setup for oxygenation treatments would be similar, except that air is introduced and removed from the device 200, instead of fluid.

The patient is positioned on a horizontal surface with the body PB lying flat on the horizontal surface. The corporeal device 200 is implanted within the body PB such that the tubular housing 100 is fully positioned within a blood vessel BV. The blood vessel BV may be either a vein or an artery. In regard to FIG. 8, the corporeal device 200 is implanted in the aorta. The tubes 202, 208, and 214 partially extend within the aorta, bisect the wall tissue of aorta, extend partially within the body cavity and penetrate a layer of muscle to connect to the respective pouch 220, 240, and 260. The pouches 220, 240, and 260 are preferably implanted between a layer of skin and the layer of muscle. The tubular housing 100 is held in place within the aorta by the internal fluid pressure of the aorta. A suture is made around the tubes 202, 208, and 214 where they exit the aorta to prevent blood loss through the opening made in the aorta.

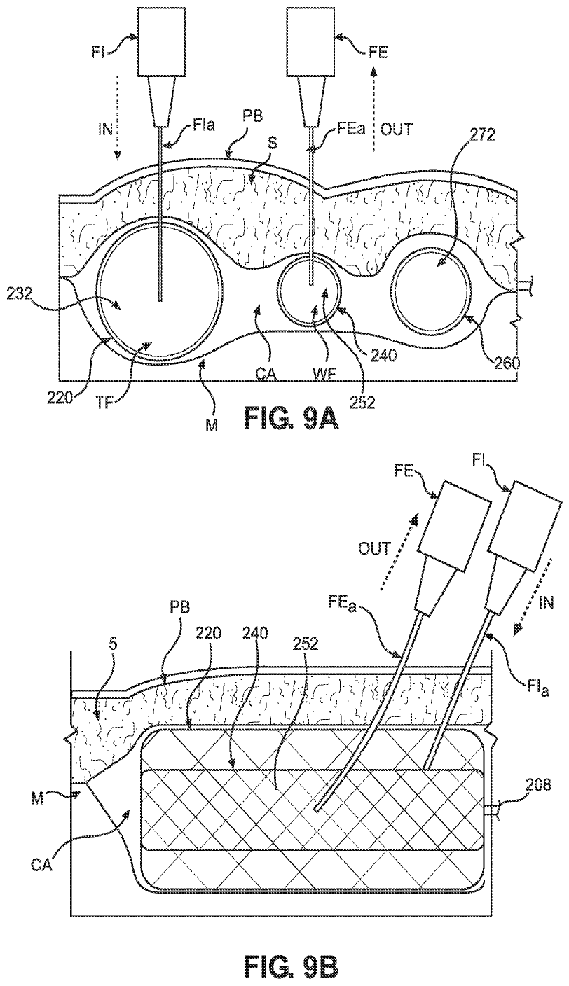

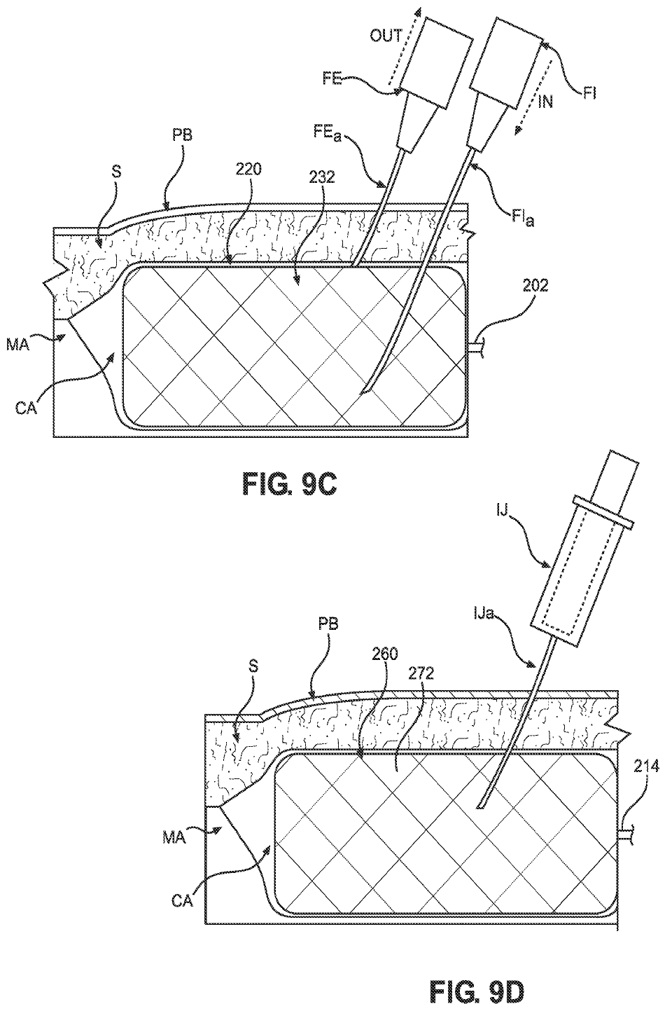

FIG. 9A shows a cross-section along the patient's body PB and along the three pouches 220, 240, and 260 provided in FIG. 8. For the purposes of this illustration, the three pouches 220, 240, and 260 are aligned such that the cross-sectional view captures all of the pouches. However, the three pouches 220, 240, and 260 need not be aligned as shown upon implantation. Referring again to FIG. 9A, the three pouches are implanted beneath a layer of skin S, between the skin and a layer of muscle M in a cavity CA. The three pouches may be positioned anywhere on the body but are most conveniently positioned along an abdomen for comfort of the patient.

The first pouch 220 has treatment fluid TF, which is introduced into the first pouch via an injector FI. A needle FIₐ or similar hollow structure attached to the injector FI pierces the skin S and the first pouch 220 and is inserted into the inner cavity 232 defined by the first pouch. Treatment fluid TF contained in a container FR₁ travels from the container through the connected injector FI and is transferable through the injector into the cavity 232 via the needle FIₐ. The treatment fluid TF then travels through the first tube 202 to the inner cavity 116 of the tubular housing 100. FIG. 9C provides a side view of the first pouch 220 and injector FI oriented according to FIG. 9A.

The second pouch 240 contains waste fluid WF, which is removed from the second pouch via an extractor FE. A needle FEₐ or similar hollow structure attached to the extractor FE pierces the skin S and the second pouch 240 and is inserted into a hollow cavity 252 defined by the second pouch. Waste fluid WF passes from the inner cavity 116, through the second tube 208, and into the hollow cavity 252, where it is removed via the needle FEₐ. The waste fluid WF then travels through the extractor FE to a waste disposal container FR₂. FIG. 9B provides a side view of the second pouch 240 and the extractor FE oriented according to FIG. 9A.

The third pouch 260 is provided for one-time introduction of materials into the corporeal device 200, such as medication, whereas the first and second pouches 220 and 240 are continuously receiving and expelling liquid via the injector FI and extractor FE. This is represented by injector IJ, which in this embodiment is a syringe and needle. The injector IJ may be other known and used devices for injecting fluids into a patient's body PB. A needle IJₐ or similar hollow structure attached to the injector IJ pierces the skin S and the third pouch 260 and is inserted into a hollow cavity 272 defined by the third pouch. Medication or similar fluid is transferable through the injector IJ into the hollow cavity 272 via the needle IJₐ. The medication then travels through the third tube 214 to the inner cavity 116 of the tubular housing 100. FIG. 9D provides a side view of the third pouch 260 and injector IJ oriented according to FIG. 9A.

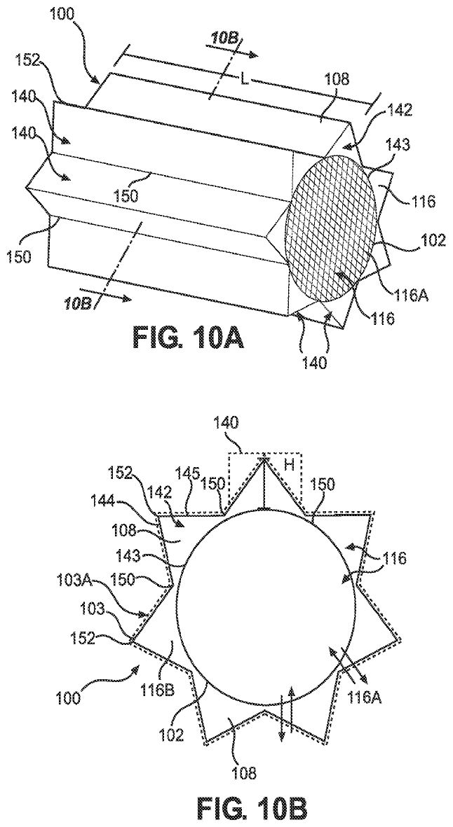

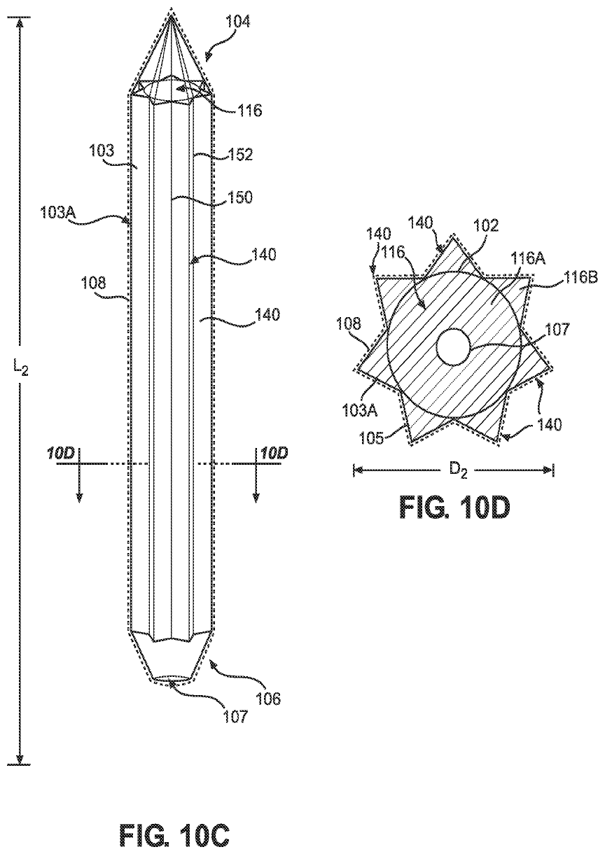

FIG. 10A depicts a section of an alternative embodiment of the tubular housing 100 having increased surface area for the semi-permeable bio-modified layer 108. This embodiment of the tubular housing 100 has a plurality of longitudinal structures 140 having a generally triangular cross-section 142, where one side 143 of the triangular cross-section is curved and not linear. The other two sides 144, 145 of each triangular cross-section have equal lengths. Each of the plurality of longitudinal structures 140 is oriented around a circumference of a tubular frame 102, and is collapsible, expandable, and hollow. The tubular frame 102 is likewise collapsible, expandable, and hollow. Each longitudinal structure has a height H, which is between 1-4 mm, and preferably 2.5 mm. FIG. 10B shows a cross-sectional view of the tubular housing 100 along plane 10B-10B.

Together, the plurality of longitudinal structures 140 and tubular frame 102 define an inner cavity 116 which is separated from an external environment by the semi-permeable bio-modified layer 108. An inner area 116B of each longitudinal structure 140 and an inner area 116A of the tubular frame 102 together form the inner cavity 116, and fluid is freely communicable between the inner areas 116a and 116b.

The longitudinal structures 140 may be formed from a single, continuous sheet 103 of material with a section formed in a circular, zig-zag pattern around the tubular frame 102. The sheet 103 should be made of a material that is porous or permeable for fluid, molecules, and/or other compounds to pass through. The sheet 103 may be attached to the tubular frame 102 along inner linear joints 150. The bio-modified layer 108 can be applied to or laid across an outer surface 103A of the sheet 103.

Alternatively, there may be a plurality of sheets 103 with widths extending between and secured together along inner linear joints 150 and outer linear joints 152. These sheets 103 may be attached together and arranged in the circular, zig-zag pattern.

Further, the sheets 103 themselves may be the semi-permeable bio-modified layer 108. In such an embodiment, the bio-modified layer 108 is not applied or laid across the outer surface 103a of the sheet 103. Instead, the bio-modified layer 108 and the sheet 103 are the same structure.

In another possible embodiment, the tubular housing 100 does not have a tubular frame 102. Instead, the sheet 103, whether a single, continuous sheet or a plurality of sheets connected together, may be rigid enough to form the cross-section show while under fluid pressure inside a blood vessel of the body, but flexible enough to axially collapse in diameter towards center axis CH, as with all the other embodiments of the tubular housing 100. When the sheet 103 is made of such a material, a tubular frame 102 may not be necessary.

FIG. 10C shows the tubular housing 100 embodiment for use with a corporeal filtration and oxygenation device, similar to the embodiment shown in FIG. 2A. When used with the device of FIG. 2A, the tubular housing 100 of FIG. 10C would replace the tubular housing 100 of FIG. 1A. A bio-modified layer 108 is shown covering an outer surface 103A of the sheet 103. The inner cavity 116 is formed within the tubular housing 100, with the bio-modified layer 108 covering an entire outer surface of the tubular housing, or at least the surface of the tubular housing that contacts blood within the body. The tubular housing 100 is closed at an end 104 by the bio-modified layer 108. However, there is an opening 107 along the end 106 to allow one or more structures, such as tubes 220, 240, and/or 260, to be inserted into the cavity 116 via the opening. In an operably expanded diameter, or the diameter after implantation and during use for filtration and/or oxygenation, the tubular housing 100 preferably has a diameter D₂ between 6-9 mm. A length L₂ of the tubular housing may vary between 152.4-355.6 mm, or 6-14″. A preferable implantation site for the tubular housing is in an aorta. The aorta in an average adult has a diameter of 18-36 mm and provides ample room for blood to flow around the tubular housing 100. Implantation in other blood vessels is contemplated, including in either arteries or in veins of sufficient size to allow sufficient blood flow around the tubular housing 100. FIG. 10D is a cross-sectional view of the tubular housing 100 of FIG. 10C across the diameter D₂ of the tubular housing at plane 10D-10D. As with other embodiments, the bio-modified layer 108 is shown in broken lines around the outside of the tubular housing to show that it is semi-permeable and covers any surface that would ordinarily have the chance of coming into contact with blood.

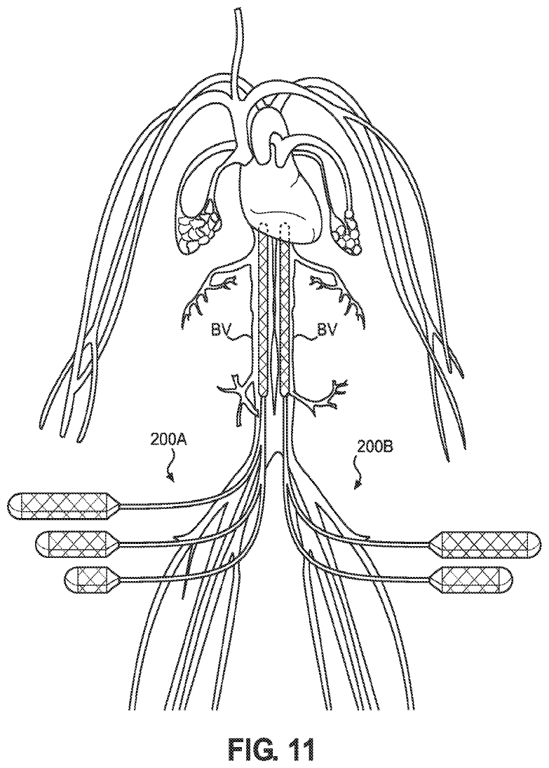

FIG. 11 illustrates two corporeal devices implanted in a body. A corporeal device 200A is implanted in an artery, namely, the aorta. A corporeal device 200B is implanted in a vein, namely, the vena cava. Each device 200A, 200B, separately or together, act as an implantable kidney dialyzer. The devices 200A, 200B may be implanted in other blood vessels, as applicable for the patient's health. The corporeal device 200B is an embodiment that includes two pouches, such as shown in FIG. 7, while the second corporeal device is an embodiment that includes three pouches, such as shown in FIG. 2A. Both embodiments could include two pouches or three pouches. Each device 200A, 200B is inserted into a respective blood vessel with each tube of the device extending from inside the blood vessel, through a wall of the blood vessel and into an interior of the body. A suture may be used to close and seal the wall of the blood vessel around the tubes. Each pouch of each device is positioned ideally in the abdomen between a layer of skin and muscle with each respective tube extending from the corresponding pouch to the tubular housing of the corresponding device. In this configuration, one device can be performing oxygenation and the other can be performing filtration.

With any of the embodiments of the device 200 described herein, the pouches 220, 240, and/or 260, including a partial length of corresponding tubes 202, 208, and/or 214, may be positioned outside of the body, while the tubular housing 100 and a remaining length of the corresponding tubes 202, 208, and/or 214 are implanted within the body, as described. In this case, the tubes 202, 208, and/or 214 bisect both the muscle M and the skin S, and the injector FI and extractor FE are connectible to the pouches 220 and 240 outside of the body in the same manner. A layer of bio-modified material would not need to cover the outer layers of the lengths of the tubes 202, 208, and/or 214 outside of the body, nor would the bio-modified layer need to cover the pouches 220, 240, and/or 260. Such structures would not be in contact with blood circulating in the body, or to be re-introduced to the body.

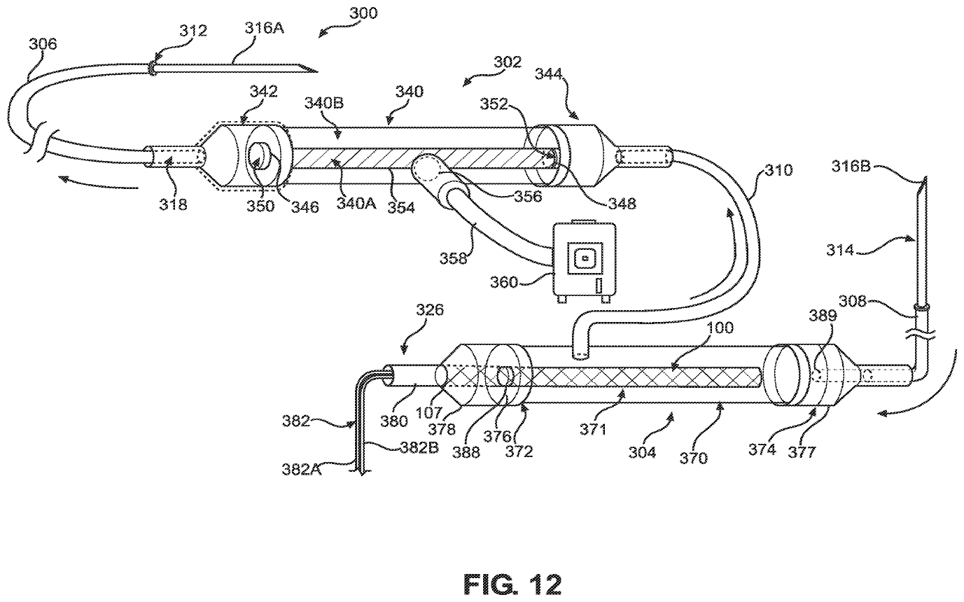

A para-corporeal embodiment of a filtration and oxygenation device 300 is shown in FIG. 12. The para-corporeal device 300 includes a pump 302 connected to a differential container 304, with a first connection pathway 306 connected to the pump and a second connection pathway 308 connected to the differential container. In this embodiment, the pump 302 and the differential container 304 are connected by a connection pathway 310. The first connection pathway 306 has a free end 312 ending in a piercing element 316A, and the second connection pathway 308 has a free end 314 ending in a piercing element 316B. The piercing elements 316A and 316B are typically needles or similarly thin hollow structures insertable into a patient's skin and through which blood can flow. The piercing elements 316A and 316B are typically made from a metal or metal alloy, as known in the art, which are non-reactive with blood. The first connection pathway 306 and second connection pathway 308 are fluidly connected to the piercing elements 316a and 316b, respectively, such that fluid is passable through the piercing element 316a and into the first connection pathway 306 and from the second connection pathway 308 into and out of the piercing element 316b.

At a longitudinal end 318, the first connection pathway 306 connects to the pump 302. Through this connection, fluid can flow from the piercing element 316a, through the first connection pathway 306, and into the pump 302.

The pump 302 includes a longitudinal housing 340 with opposing ends 342 and 344. The housing 340 is closed at each end 342 and 344 except for an opening 350 along end 342 and an opening 352 along end 344. An openable and closeable valve 346 is positioned within opening 350, and another openable and closeable valve 348 is positioned within opening 352. The valve 346 controls fluid flow from the first connection pathway 306 into the pump 302, and the valve 348 controls fluid flow from the pump to the connection pathway 310. A flexible tubular bridge 354 is positioned within the housing 340, defines a pathway between the valves 346 and 348, and defines a fluid chamber 340a and an air chamber 340b within the housing 340. The housing 340 has an opening 356 along its length. The opening 356 is attached to an air pump 360 via tubing 358 or similar air-tight connection for supplying and removing air from within the air chamber 340b.

The differential container 304 includes a housing 370 defining an inner volume or cavity 371 between two opposing longitudinal walls 376 and 377 at longitudinal ends 372 and 374, respectively. An inner surface 371S of the cavity 371, including walls 376 and 377 and the housing surfaces in the cavity, are covered by a bio-modified layer that does not cause an immunological insult to blood when the two materials come into contact. Preferably, the inner surface 371S is covered in pericardium. However, similar biological tissues or materials, whether natural or synthetic, may be used as long as an immune response, as described herein, is not initiated due to blood contact with such tissues or materials.

Wall 376 has an opening 388 through which a partial length of a tubular housing 100 extends. The opening 388 is sealed around the tubular housing 100 such that fluid cannot escape the cavity 371 through the opening. Wall 377 has an opening 389. The connection pathway 308 is attachable to the end 374 of the differential chamber 370 such that the opening 389 is secured to the connection pathway 308 and fluid is communicable from the cavity 371, through the opening 389, and into the cavity the connection pathway 308. The opening 107 of the tubular housing 100 is in fluid communication with one or more tubes 382 via an element 380. The element 380 is attached to the one or more tubes 382, which may be similar to tubes 202, 208, and 214, and the element 380 is removably attachable to a housing portion 378 extending beyond the wall 376. The tubes 382 extend into an inner cavity 116 of the tubular housing 100, which can be modeled on the tubular housing 100 of FIG. 1A or FIG. 10C. Each tube 382 may extend along different lengths of the tubular housing, similar to the tubes 202, 208, 214 of the corporeal device 200. At opposing ends, the tubes are connected to various structures, depending on the application of the para-corporeal device 300.

For instance, if used for oxygenation, one tube 382A is connected to an oxygen tank or supply and carries oxygen from the supply into the tubular housing 100. The other tube 382b carries air from the tubular housing 100 to an external environment or an air deposit. If the device is used for filtration, the tube 382a can supply treatment fluid to the tubular housing 100, while the tube 382b removes waste fluid from the tubular housing.

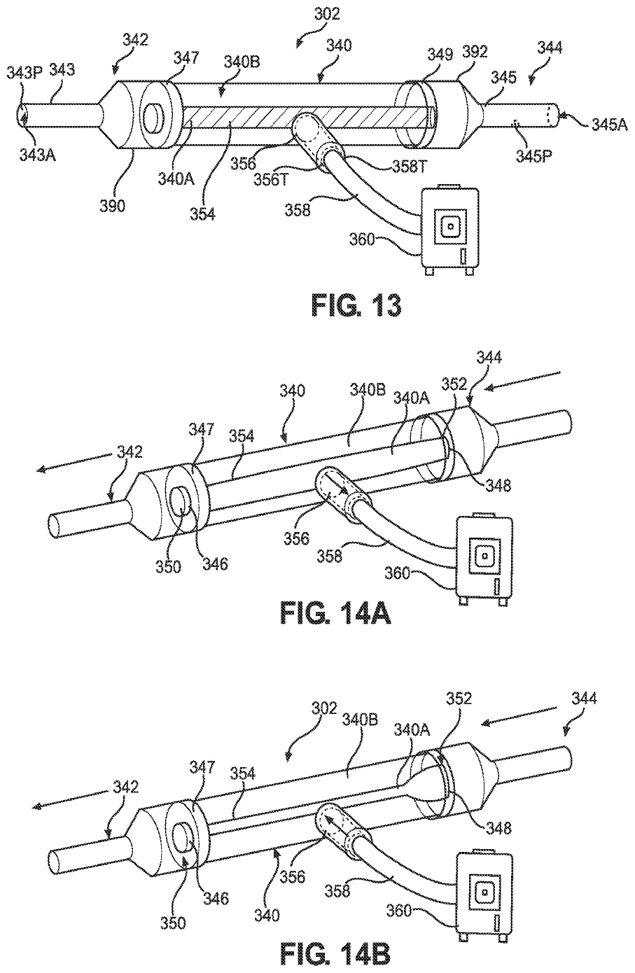

FIG. 13 provides an illustration of the pump 302 without the first connection pathway 306 and connection pathway 310 attached. A wall 347 along end 342 and a wall 349 along end 344, along with the housing 340, defines the air chamber 340B around the tubular bridge 354. On a side of the wall 347 opposite to the air chamber 340B extends a hollow connection member 343 with an opening 343A. An inner surface 343P of the hollow connection 343 is covered in or made of bio-modified layer, preferably pericardial tissue. Likewise, a hollow connection member 345 extends from the wall 349 on a side opposite from the air chamber 340B. The hollow connection member 345 has an opening 345A and has an inner surface 345P covered in or made of a bio-modified layer, preferably pericardial tissue. Both surfaces 343P and 345P may be made of or covered in other biological tissue or material that is meant as, or mimics, a blood-contacting surface that does not create an immunological insult. The members 343 and 345 are connectable to tubes or other hollow structures for transporting liquids, including the connection pathways 306 and 310, via openings 343a and 345a, respectively. Such tubes, like connection pathways 306 and 310, may be connectable directly to openings 350 and 352, or they may be connectable to a respective housing 390, 392 located at ends 342, 344. If the housings 390, 392 are present, an inner surface of the housings must also be covered in bio-modified material. Each housing fluidly connects the tubes 306, 310 to the tubular bridge 354 to direct fluid flow into and out of the pump.

The valves 346 and 348 are openable and closable to allow or prevent fluid flow past each respective valve. The valves 346, 348 are preferably alternatively openable and closeable, such that when valve 346 is open, valve 348 is closed, and vice versa.

The opening 356 may include a threaded section 356T that corresponds to a threaded section 358t of the tubing 358 connecting the pump 302 to the air pump 360. This allows the tubing 358 to removably and threadingly engage the opening 356.

An entire inner surface of the flexible tubular bridge 354 and each valve 346 and 348 are likewise made of or covered in a bio-modified layer that is meant as, or mimics, a blood-contacting surface. It is important that any surface that blood could come into contact with, in relation to the device 300, is made of or covered in a material that will not initiate an immunological response or constitute an immunological insult.

FIGS. 14A and 14B illustrate how the pump 302 moves blood, or any other fluid, through the pump in alternating diastole and systole phases. The pump 302 is in the diastole phase in FIG. 14A. The valve 346 closes, preventing fluid flow out of the end 342. The air pump 360 removes air from the air chamber 340B, or at least allows pressure in the air chamber to decrease, via the tubing 358 and opening 356. Air exiting the air chamber 340B is shown by an arrow in tubing 358. Removing air from the air chamber 340B reduces counteracting pressure on the flexible tubular bridge 354. This allows fluid to fill the tubular bridge 354, which expands due to pressure from the fluid inside pushing outwards due to the lack of counter-acting air pressure in the air chamber 340B. Fluid enters the tubular bridge 354 via the opening 352 and the open valve 348.

In the systole phase, shown in FIG. 14B, the air pump 360 adds air to the air chamber 340B, shown by the arrow now going in the opposite direction out of the tubing 358. In response, the valve 348 closes the opening 352. The air increases pressure in the air chamber 340B, and the pressure eventually exceeds the outward fluid pressure in the fluid chamber 340A acting on the tubular bridge 354. This air pressure squeezes the flexible tubular bridge 354 and reduces the volume of the fluid chamber 340A. The valve 346 opens in response and fluid inside the fluid chamber 340A exits through the opening 350 and leaves the pump 302 via end 344.

Cyclically removing and adding air to the air chamber 340B via the air pump 360 creates an alternating cycle of diastole and systole phases in the pump 302 to fill and empty the fluid chamber 340A to move fluid in one direction through the pump. These diastole and systole phases in the pump 302 can be synced with the diastole and systole phases of the patient's body.

In some embodiments of the pump 302, the flexible tubular bridge 354 can be configured to be permeable to pressurized oxygen. In such an embodiment, instead of the air pump 360 adding and removing air to the chamber 340B, the air pump instead removes and adds oxygen to create the cycle of diastole and systole phases to move blood through the pump 302. Additionally, oxygen can permeate the tubular bridge 354 to oxygenate the blood as it is moved through the pump 302. In such an embodiment of the para-corporeal device 300, filtration can occur in the differential device 304 while oxygenation occurs in the pump 302.

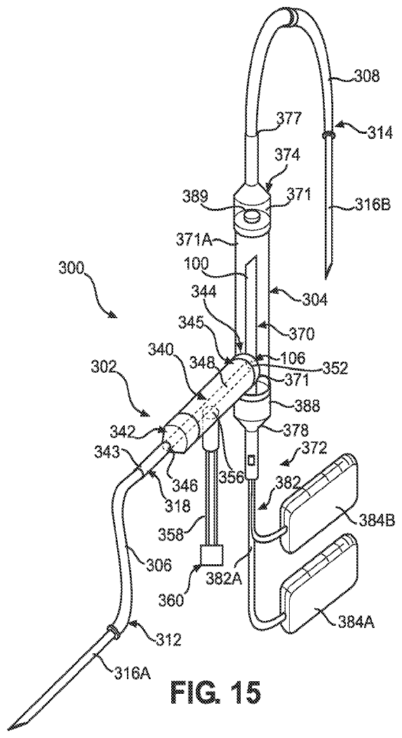

An alternate embodiment of the para-corporeal device 300 is shown in FIG. 15. Instead of the pump 302 and differential chamber 370 being connected by connection pathway 310, the pump and differential chamber are directly attached to each other. Along end 344 of the pump 302, the opening 352 leads directly into the cavity 371 of the housing 370 of the differential chamber 304. This allows fluid in the cavity 371 to flow directly from the differential device to the pump through the opening 352 along the valve 348, and into the tubular bridge 354. Connection member 345 is removed and the pump 302 is connected directly to the differential chamber 370 along the end 344. The differential chamber 370 may be sized to create different inner volumes, depending on the application of the device 300 and the patient's needs.

Further, this embodiment of the device 300 is configured for filtration treatment. Therefore, a pouch 384A is attached to tube 382A, and a pouch 384B is attached to tube 382B. The tubes 382 could also be attached to oxygen-in lines and air out lines, similar to the device 300 of FIG. 12, and vice versa. The remaining elements of the device 300 shown in FIG. 15 are the same as the embodiment shown in FIG. 12.

In all embodiments of the para-corporeal device 300, the connection pathways 306, 308, and/or 310 each have an inner surface which is covered in a biological material that does not cause an immunological insult to blood when the two materials come into contact. Preferably, the inner surfaces are covered in pericardium. However, similar biological tissues or materials, whether natural or synthetic, may be used as long as an immune response, as described herein, is not initiated due to blood contact with such tissues or materials.

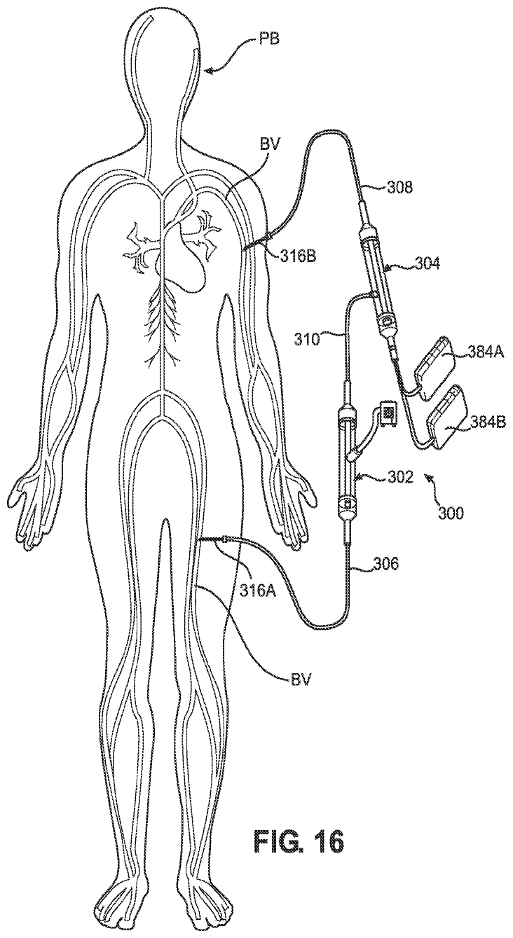

FIG. 16 illustrates a representation of a human body with the para-corporeal device 300 attached for treatment. In this case, the device 300 is in the form of the embodiment shown in FIG. 12, except that it has two pouches attached for filtration purposes.

The piercing element 316B is inserted into a blood vessel, which in this case is the Subclavian vein. The piercing element 316a is inserted into another blood vessel, which in this case is the Femoral artery. Other veins and arteries may be used. Blood enters the device 300 through the piercing element 316b and travels through the connection pathway 308, and into the cavity 371 of the differential chamber 304. The blood comes into contact with the bio-modified layer 108 of the tubular housing 100 longitudinally extending within the cavity 371 of the differential chamber. Preferably, the housing 370 and tubular housing 100 are coaxial and share the axis CH. This allows blood to contact the entire outer surface of the portion of the tubular housing 100 extending within the cavity 371.

The pouches 384A and 384B are fluidly connected to the tubular housing 100 to provide treatment fluid TF to and remove waste fluid WF from the cavity 116 of the tubular housing. The bio-modified layer 108 of the tubular housing 100 is semi-permeable to allow certain compounds and molecules to pass from the blood to the inner cavity 116 and vice versa. This allows the blood to be filtered along parameters chosen by a healthcare professional, with the treatment fluid composition being alterable to enact such parameters.

Oxygenation of the blood is carried out in a similar manner. Instead of treatment fluid and waste fluid being added and removed from the tubular housing, oxygenated and deoxygenated air is added and removed, respectively. The oxygen is permeable across the bio-modified layer. Further, an oxygenation setup of the device 300 may not require pouches 384A and 384B, and the tubes 382 may simply be connected to an oxygenator or similar device.

The filtered and/or oxygenated blood then moves from the differential chamber 371 to the pump 302. This occurs either across the connection pathway 310 or directly, as shown in the device embodiments of FIGS. 12 and 15. Through the alternating diastole and systole phases of the pump 302, as previously described, blood is pushed from the pump 302 into the connection pathway 306 and back into the body through the piercing element 316a inserted in the Femoral artery.

While the atrio-venous connection shown in FIG. 16 is possible, such connections are stressful on the patient's body, as arterial and venous tissue are adapted to function under different pressures. By directly connecting an artery and a vein with the device 300, normal pressure in the attached blood vessels is thrown off at the insertion areas. This causes tissue damage and other complications.

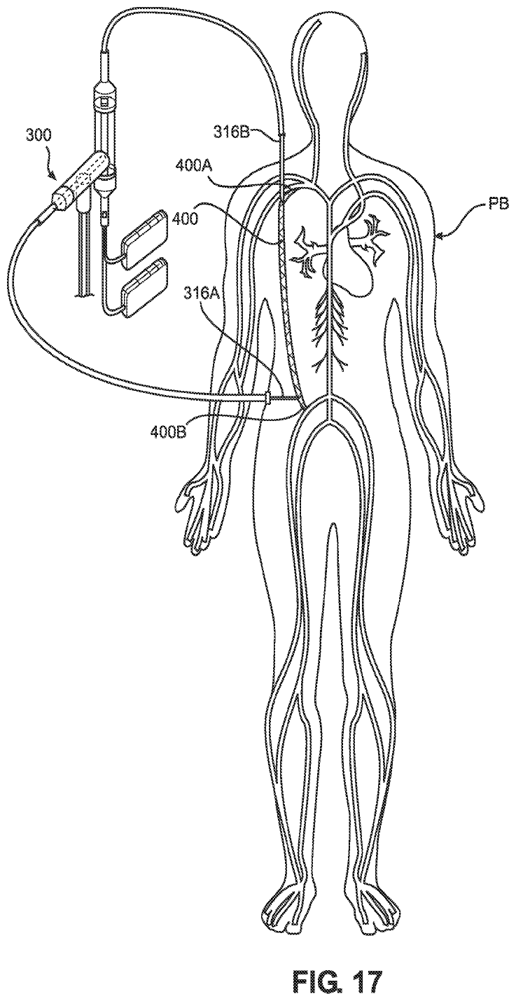

FIG. 17 shows an artery-to-artery connection for the para-corporeal device 300. A longitudinal graft 400 of biological tissue, pericardium for example, is attached to an artery at either opposing end 400A and end 400B. In this illustration, end 400A is attached to a superior artery, or an artery that supplies blood to organs and tissues above the heart, while end 400B is attached to an inferior artery, which supplies blood to organs and tissues below the heart. This graft 400 is surgically implanted and positioned within the body PB. The device 300 is then attachable to the graft 400 along both piercing elements 316A and 316B to tissue under very similar, if not identical, pressure. The use of this graft is especially beneficial for filtration of blood. The graft 400 can be attached to appropriate sets of superior and inferior arteries by sutures known in the art.

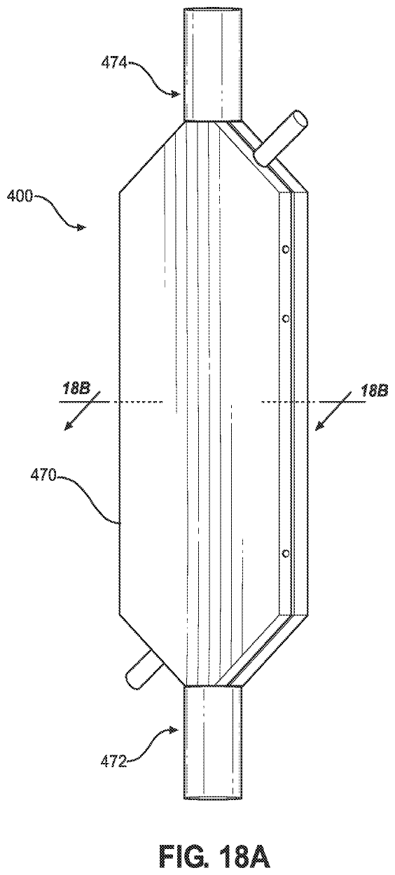

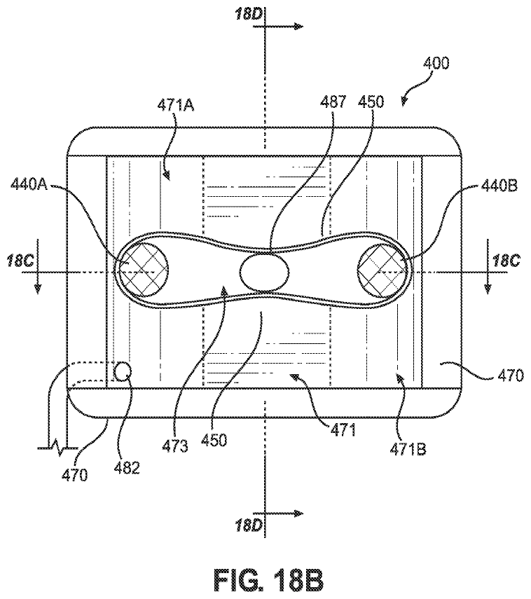

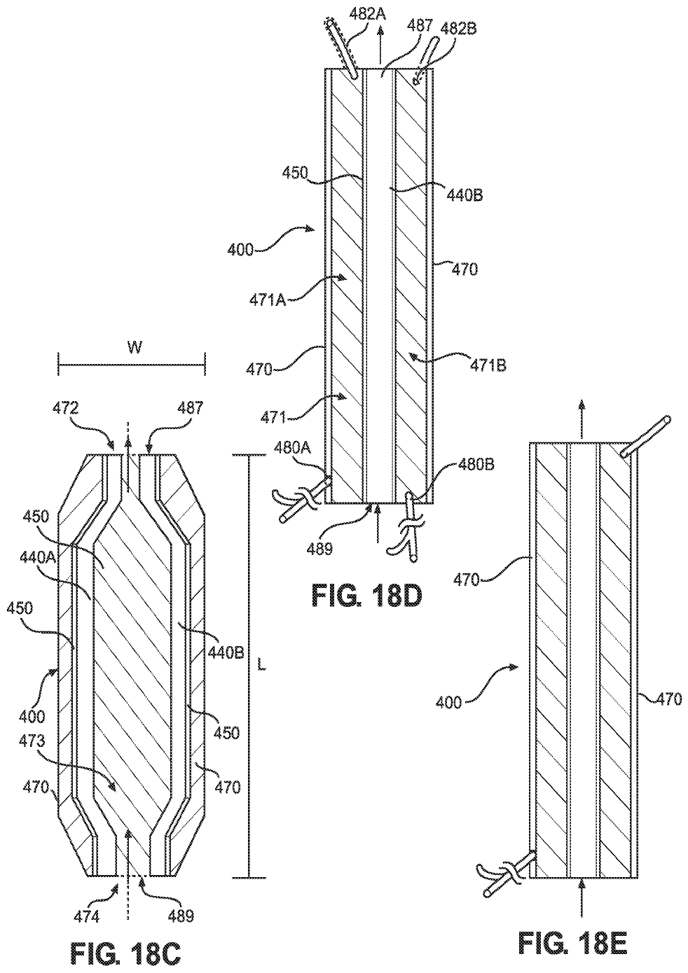

An alternative embodiment of a differential device 400 is shown in FIGS. 18A-18E. The differential device 400 is structured differently than the differential device 304 of FIGS. 12 and 15, but provides the same functions, namely, filtration and oxygenation of bodily fluids. The differential device 400 has an elongated housing 470 with ends 472 and 474 connectable to tubing or other devices, similar to the differential device 304. The housing 470 forms and defines an inner cavity 471. Two rods 440A and 440B are positioned within the inner cavity 471. The two rods 440A and 440B are vertically centered in the cavity 471 but are laterally offset. A bio-modified layer 450 is wrapped around the rods 440A and 440B such that a fluid cavity 473 is formed between the rods and the bio-modified layer. This fluid cavity is fluidly separated from the inner cavity 471. However, the bio-modified layer is semi-permeable, and differentials between the cavity 471 and chamber 473 can cause select materials to permeate in either direction across the layer 450. The chamber 473 is connected to, and sealed around, opposing openings 487 and 489 in the housing 470, such that fluid can only enter or escape the chamber via these openings. The housing 470 may have one or more material introduction openings 480 and one or more material removal openings 482, each connected to a corresponding tube or connection for transporting fluid and/or gas. Such openings 480 and 482 are connected to and fluidly communicate with the cavity 471, but not the chamber 473.

During filtration or oxygenation, blood enters the chamber 473 via opening 489 at end 474. The blood passes through the chamber 473 along a length L of the device 402 and out opening 487 along end 472. Other fluids or gases can be introduced into the cavity 471 while blood is in the chamber 473 via openings 480. Such fluid or gases can be removed from the chamber 471 via openings 482. While the fluid or gases are in the cavity 471, and blood is in the chamber 473, differentials between the matter in the cavity and the chamber can cause materials to selectively permeate across the layer 450 to filter or oxygenate the blood in the chamber 473, or to introduce medicine into the blood.

The rods 440A and 440B and layer 450 may extend along an entire width W of the cavity 471, such that an upper cavity 471A and a lower cavity 471B are formed. In such embodiments, shown in FIG. 18D, upper openings 480A and 482A and lower openings 480B and 482B may be used to introduce and remove materials in both the upper and the lower cavity. In other embodiments, the rods 440A and 440B and layer 450 may not completely bisect the cavity 471, and only one set of openings 480 and 482 are needed. Optionally, more than one set of openings 480 and 482 may be used even if the cavity 471 is not completely bisected.

The rods 440A and 440B and the layer 450 are made of, or covered by, a bio-modified layer or other biological tissue, such as pericardium, which does not create an immunological insult when contacted with blood that is reintroduced into the patient's body. Any other surface of the device 400 that could be potentially contacted by blood should likewise be covered in a similar bio-modified layer or tissue to prevent immunological responses due to immunological insults, as consistent with all other devices described herein.

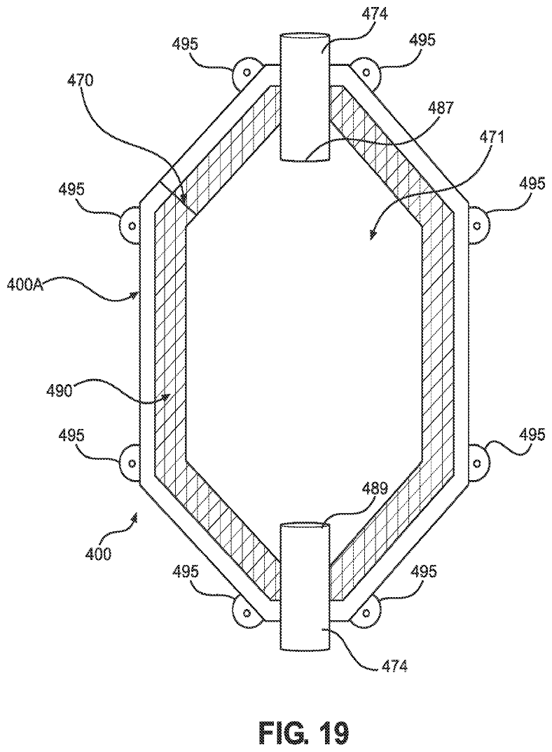

FIG. 19 is a top plan view of an alternative embodiment of the differential device 400 of FIGS. 18A-18E. In this embodiment, the device 400 includes two halves that are connected together along corresponding members 495. One half of device 400 is shown in FIG. 19 as device 400A. The device 400A has a housing 470 that includes a strip 490 of medical grade rubber. When the two halves of the device 400 are combined, they are combined such that two strips 490 are pressed against each other to form the cavity 471. In one of the halves 400A, the ends 474 and 472 each include a section of tubing extending inside and outside of the cavity 471. These tubes can be molded into the rubber of the strip 490, with the other half of the device having a corresponding indent to accommodate the tubes when secured to the other half of the device.

When the halves of the device are secured together, the bio-modified layer 450 can be partially secured between the two halves and between the two strips 490. If necessary, the strips 490 may also be covered in a separate bio-modified layer. When forming the fluid cavity 473, the tubes inside the cavity 471 corresponding with each end 472 and 474 should extend into the fluid cavity.

The rods 440A and 440B and layer 450 are not shown in FIG. 19, but otherwise are included as provided in the device 400 of FIGS. 18A-18E. The differential device 400 of FIG. 19 operates similarly to the device 400 of FIGS. 18A-18E, with the main difference being the housing 470 formed of two halves each with a strip 490 of medical grade rubber.

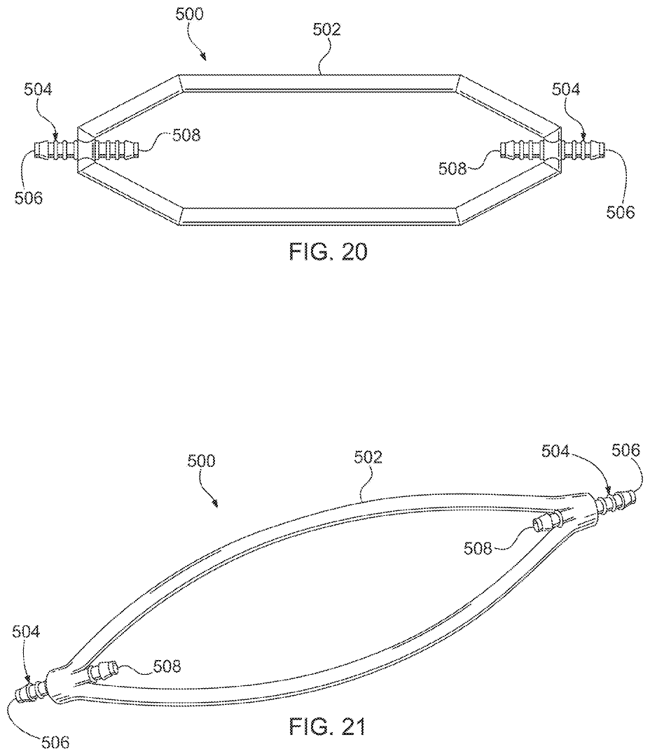

FIG. 20 shows a tissue frame 500 embodiment for use with a differential device 400 or 600. The tissue frame 500 has a member 502 that is continuous to form a loop or similar structure. The member 502 may be rigid, semi-flexible or flexible. The loop formed by the member 502 is elongated in FIG. 20, with defined linear lengths. However, the loop may be perfectly circular or otherwise shaped to conform to a corresponding differential device. Two hollow cylindrical members 504 extend through openings 514 in the member 502, with one hollow cylindrical member extending through one opening. Each hollow cylindrical member 504 has exterior opening 506 and an interior opening 508, with each interior opening positioned within the loop and each exterior opening positioned outside of the loop. Fluid can pass within and through, in either direction, each hollow cylindrical member 504 via the openings 506 and 508.

FIG. 21 shows an alternate embodiment of the tissue frame 500. In that embodiment, the loop formed by the member 502 does not have linear segments or at least fewer linear segments. Instead, the loop is curved on either side. As previously stated, the exact shape of the loop may be changed to conform to a corresponding differential device 400 or 600.

For all embodiments of the tissue frame 500, it is important that any junctures between each hollow cylindrical member 504 and the member 502 are fluidly sealed such that blood, water, or other fluids commonly used in medical settings cannot leak between compartments of the differential device 400 or 600 or out of the differential device. The member 502 can be manufactured from a single piece of material, ideally silicon-based, or from multiple pieces attached together. However, again, it is important that the resulting member 502 is properly shaped to conform to the corresponding differential device 400 or 600 and that any junctures or attachment points between pieces of the member are fluidly sealed to ensure proper differential movement across semi-permeable membranes.

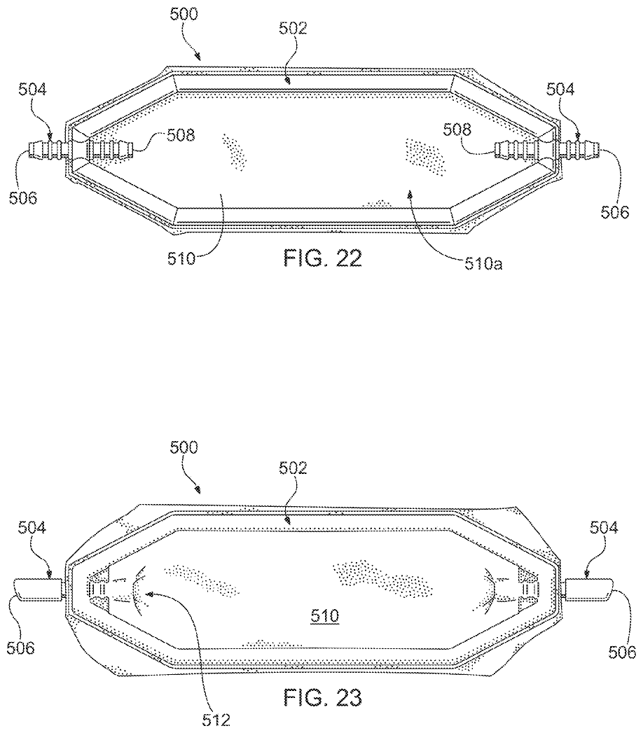

FIG. 22 shows a tissue frame 500, in this case shaped as provided in FIG. 20, with a tissue layer 510 attached to one side of the member 502. Both hollow cylindrical members 504 are positioned on the same side of the tissue layer 510. In this configuration, the tissue frame 500 is configured to provide one differential chamber on the opposite side of the tissue layer (not shown) while the side 510a shown is configured to act as the primary chamber for recirculated fluid (i.e. fluid that has been removed from a body, or other closed system, and will subsequently be reintroduced to the body after filtration or oxygenation).

The tissue frame 500 alone does not define the differential or primary chambers. The tissue layers 510, together with differential device housing, define the differential or primary chambers. The member 502 acts as a frame for the tissue layer 510, while differential housing, described in more detail in relation to differential device 600, acts as a holder for the member and tissue layer.

FIG. 23 shows a tissue frame 500 embodiment encased in tissue layer 510. Typically, the encasement of the tissue frame 500 is formed by two separate tissue layers 510 with the member 502 placed there between. The two tissue layers 510 are then sealed either directly to the member 502 or to each other along an outside of the member 502. This creates a sealed, primary chamber 512 within the loop formed by the member 502 and between the two tissue layers 510. The only pathway for fluid to freely enter or exit the chamber 512 is through either hollow cylindrical member 504. The tissue layers 500 are preferably made from, or coated with, a bio-modified material that does not induce an inflammatory response in a patient due to contact with blood or other biological material reintroduced into the patient's body.

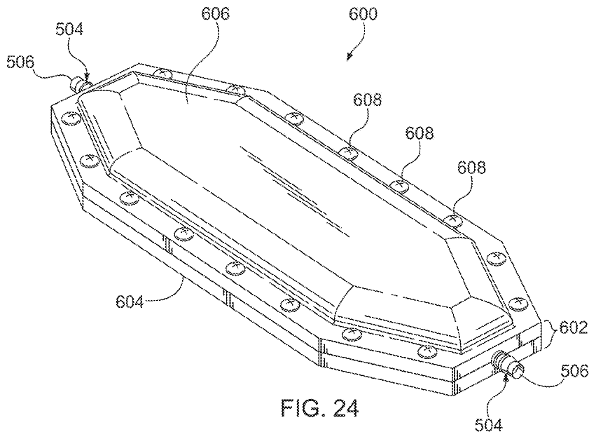

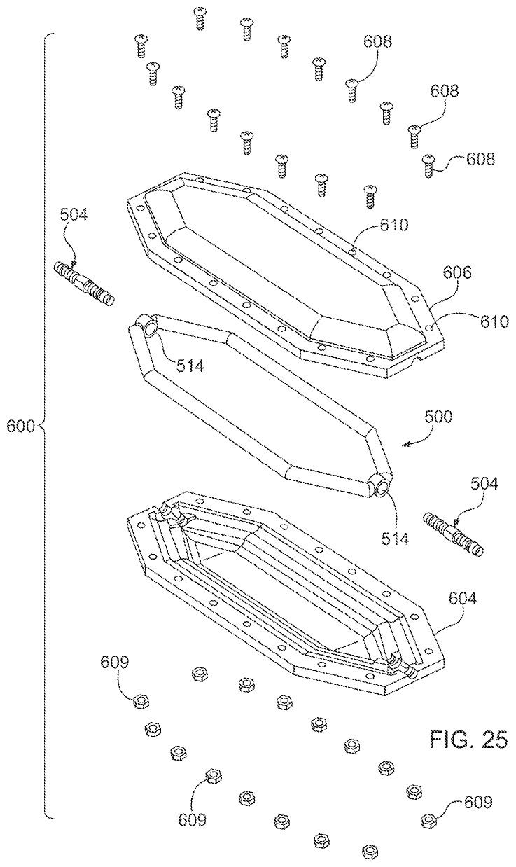

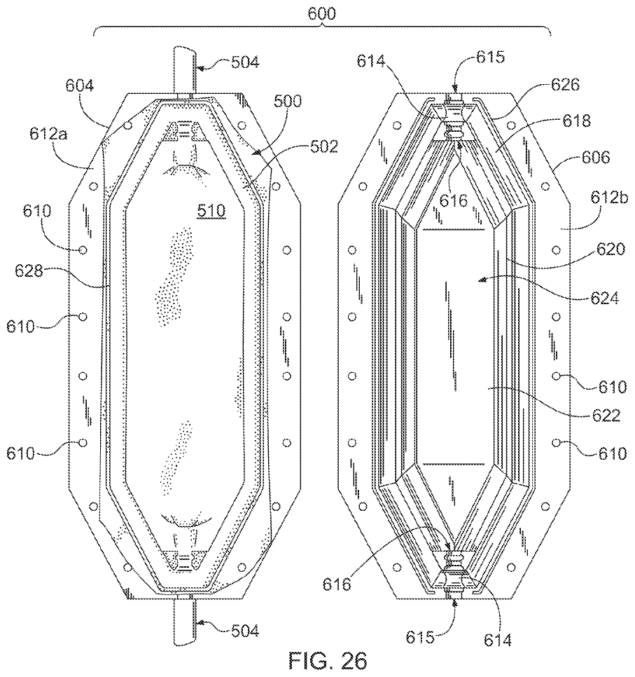

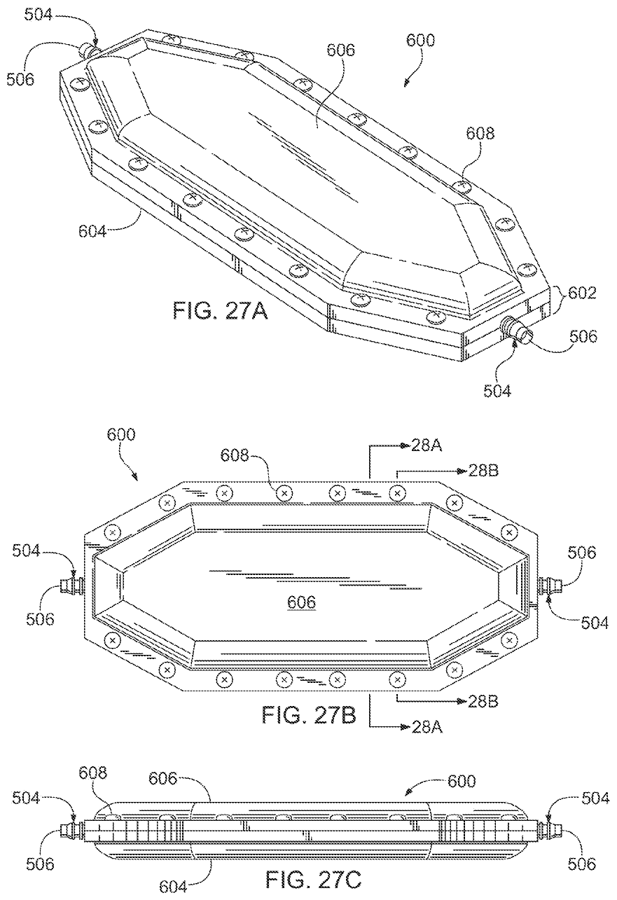

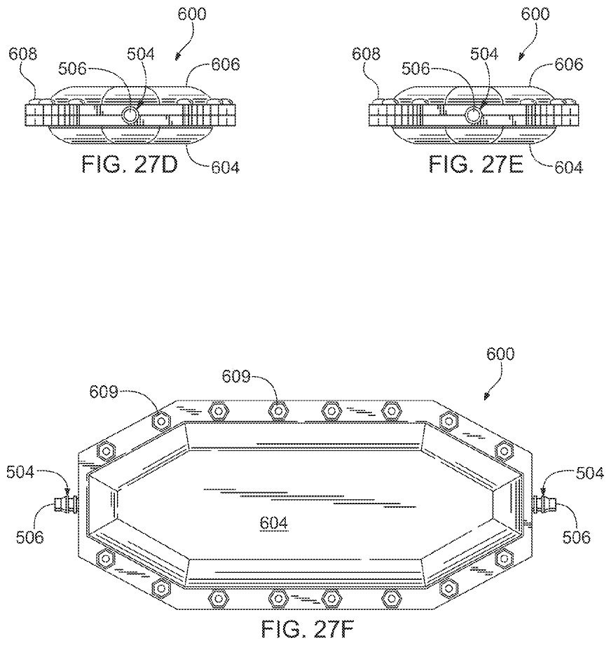

FIGS. 24, 25, 26, 27A-27F, and 28A-28B show an embodiment of a differential device 600. Like differential device 400, the differential device 600 provides a volume in which a differential is created between two different liquids and/or gases across a semi-permeable membrane. The differential produced is preferably used to either oxygenate or filter blood.

The differential device 600 includes a housing 602, having two halves 604 and 606, and the tissue frame 500 having at least one tissue layer 500 secured between the two halves of the housing. The halves 604 and 606 of the housing 602 are secured together to form a complete fluid seal such that no fluid leaks between the two halves of the housing. In this embodiment, this is achieved through a plurality of corresponding fasteners being nuts 609 and bolts 608 secured through corresponding holes 610 in both halves 604 and 606. Surface 612A of half 604 is matched up with surface 612B of half 606 such that the holes 610 of half 604 match up and are continuous with holes 610 if half 606.

Each half 604 and 606 has groove 618 extending along an inner surface, the groove 618 corresponding to and configured to hold a partial circumference of the member 502 and cylindrical member 504 of the tissue frame 500. Each half 604 and 606 also has a pair of grooves 614, groove 614 bisecting the grove 618 and having an end 615 adjacent to an outer surface of the respective half 604 or 606 and an end 616 adjacent to an interior cavity 622 defining a fluid space 624. The grooves 614 of halves 604 and 606 are shaped so that, together, they conform to the shape of the corresponding hollow cylindrical member 504, and member 502 around opening 514, when inserted in the member 502. Likewise, the grooves 618 of the halves 604 and 606, together, conform the shape of the member 502. Each hollow cylindrical member 504 extends out of the housing 602 past ends 615 and extends past ends 616 into the interior cavity 622.

To aid with lining up the halves 604 and 606 to secure them together, half 604 has a female groove 628 extending along and into the surface 612A and along the outside of the groove 618, and half 606 has a corresponding male member 626 extending along and out of the surface 612B for insertion into the groove 628.

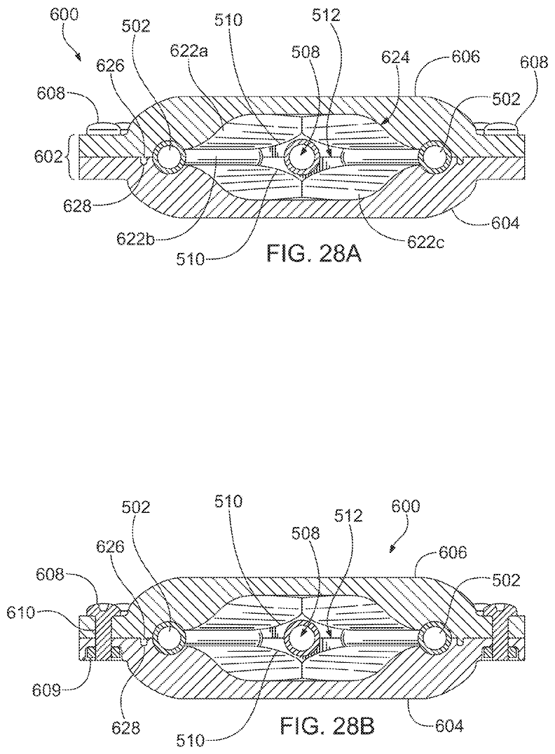

Looking at the cross-sections of the differential device 600 of FIGS. 28A-28B, the tissue frame 500 is inserted and secured in the housing 602 between halves 604 and 606. The tissue frame 500 is encased with tissue 510 to create the primary chamber 512 within the tissue frame, which corresponds to fluid chamber 622B of the differential device 600. The tissue 510 also creates a fluid chamber 622A and 622C on either side of the fluid chamber 622B. The fluid chambers 622A and 622C are fluidly sealed between the surface of the interior cavity 622 of respective halves 604 and 606 and the tissue layer 510. However, the tissue layer 510 is semi-permeable to select molecules, compounds, materials, etc. to allow such differential matter to pass between fluid chambers 622A, 622B, and 622C.

Blood, or other fluid that it is desired to remove or add materials to, enters the differential device through the opening 506 of one of the hollow cylindrical members 504 and into the fluid chamber 622B via opening 508 of the same hollow cylindrical member 504. The blood flows in one direction through the fluid chamber 622B to the opening 508 of the other hollow cylindrical member 504 and out of the differential device 600 through the opening 506 of that other hollow cylindrical member 504. While the blood is in fluid chamber 622B or 512, matter is added to or removed from the blood via one or more differentials created across the semi-permeable membrane of the tissue 510.

Other fluids are housed in fluid chambers 622A and 622C to create the desired differentials. The fluids may have water, oxygen, salt, sugar, medicines, etc. to add or remove the same to the blood.

Alternatively, the housing 602 may have two halves 604 and 606, which after having the tissue frame 500 and fluids inserted, permanently sealed together. Alternatively, the two halves 604 and 606 may be hingedly attached together or slidably attached together or may be similarly attached with or without fasteners. The device 600 may be preloaded with fluid in fluid chambers 622A and/or 622C, such that the device is configured to be used once and then discarded (i.e., a single-use device). The device 600 may otherwise be configured to be reusable, where the fluid in the fluid chambers 622A and/or 622C is removable and replaceable after each use (i.e., a multi-use device).

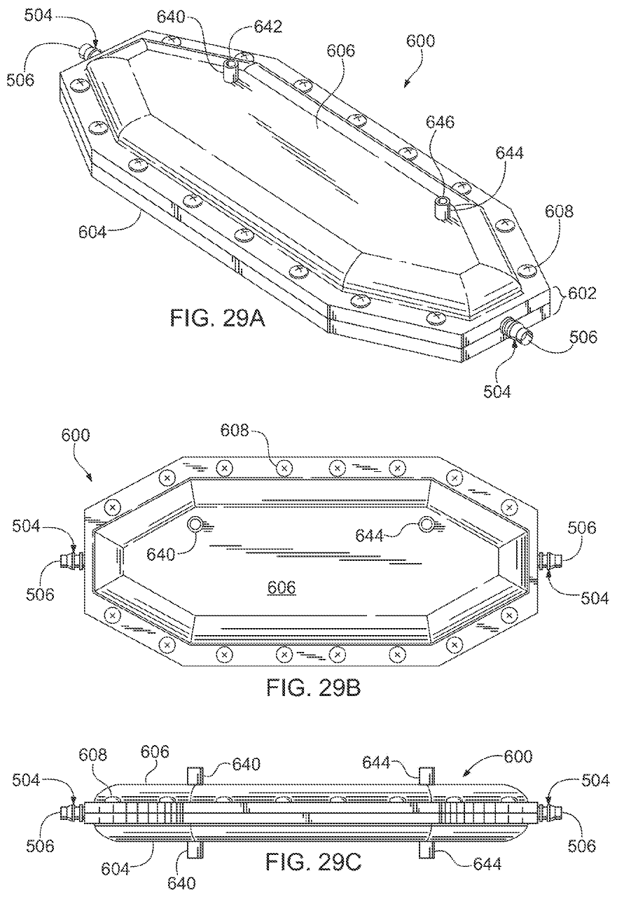

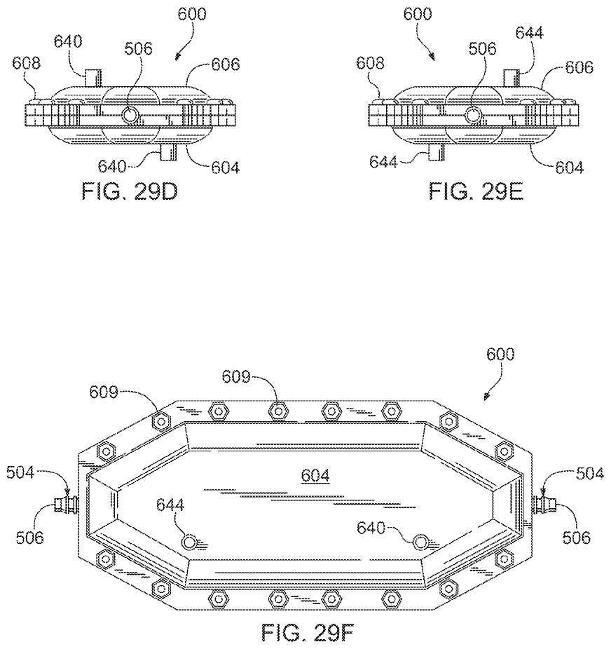

Another embodiment of the device 600 is shown in FIGS. 29A-29F. The elements and configuration of the device 600 of the previous embodiment are also present and applicable to the device 600 embodiment of FIGS. 29A-29F. However, the device 600 embodiment of FIGS. 29A-29F additionally has an intake port 640 and an output port 644 on each half 604 and 606 of the housing. When the halves 604 and 606 are secured together, each input port 640 and output port 644 fluidly connects an environment external to the device 600 to the fluid space 624, typically via tubing or similar structures.

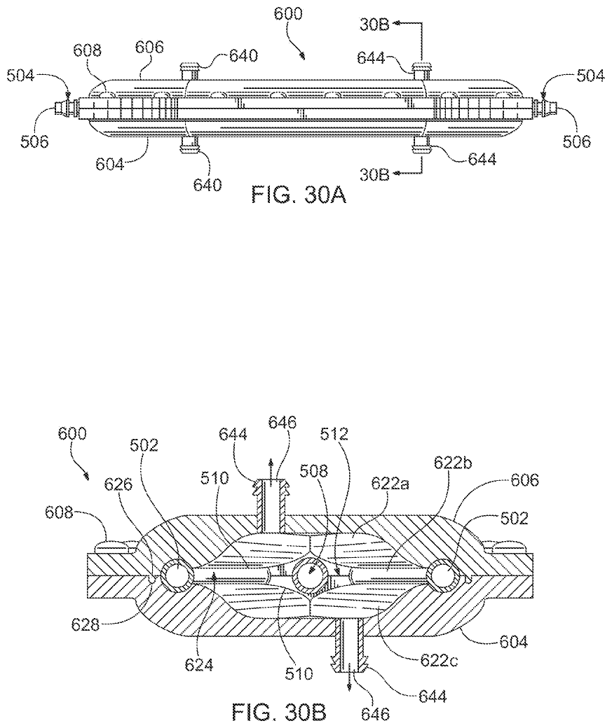

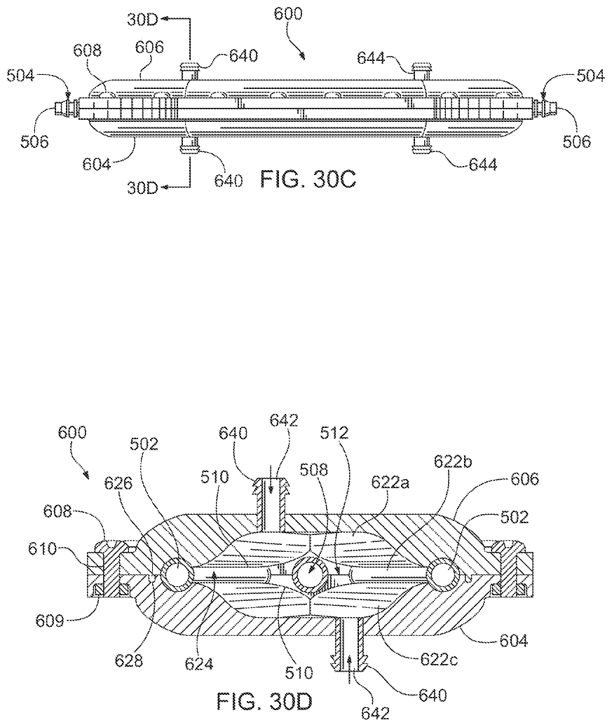

Each pair of input port 640 and output port 644 allow fluid to be circulated into and out of the device 600. FIGS. 30A-30D show an embodiment of the device 600 substantially similar to the device 600 of FIGS. 29A-29F and show how fluid would flow in and out of the device 600. The only difference is the input ports 640 and output ports 644 of FIGS. 30A-30D are flanged for helping to hold tubing around and secured to the ports 640 and 644.

For fluid half 606, fluid enters input port 640 through an opening 642 and flows into the fluid chamber 622A. The fluid flows in one direction to output port 644 and out of the device 600 through opening 646. As the fluid flows within fluid chamber 622A between ports 640 and 644, materials can diffuse between chambers 622A and 622B across the tissue.

For fluid half 604, fluid enters input port 640 through an opening 642 and flows into the fluid chamber 622C. The fluid flows in one direction to output port 644 and out of the device 600 through opening 646. As the fluid flows within fluid chamber 622C between ports 640 and 644, materials can diffuse between chambers 622C and 622B across the tissue.

The fluid flow through chambers 622A and/or 622C can be in the same direction or in the opposite direction to the fluid flow through chamber 622B. Further, this configuration of the device 600 allows for simultaneous oxygenation and filtration of the blood, with each process taking place in a different chamber 622A or 622C between chamber 622B.

The fluid chambers 622A and 622C can correspond to either half 604 or 606 of the housing 602.

If materials are to be added to blood or other primary fluid in fluid chamber 622B, material-rich fluid (or fluid with the materials present) is introduced through input port 640 and into fluid chamber 622A or 622C. As the material-rich fluid moves through fluid chamber 622A/622C toward output port 644, the material diffuses into fluid chamber 622B across the tissue 510. The fluid in chamber 622B becomes material-rich, while the fluid in 622A/622C becomes material-poor fluid (the fluid has less materials present than before it was introduced into the device 600). As fluid leaves output port 644, the fluid is preferably material-depleted (all or nearly all of the material is gone from the fluid). Alternatively, the fluid is merely material poor as it leaves the device 600 through output port 644.

If materials are to be removed from blood or other primary fluid in fluid chamber 622B, material-depleted fluid, or alternatively material-poor fluid, is introduced through input port 640 and into fluid chamber 622A/622C. As the material-depleted fluid moves through fluid chamber 622A/622C toward output port 644, material diffuses into fluid chamber 622A/622C across the tissue 510 from the material-rich fluid of chamber 622B. The fluid in chamber 622B becomes material-poor or material-depleted, while the fluid in 622A/622C becomes material-rich fluid (the fluid has more material present than before it was introduced into the device 600).

The device 600 of FIGS. 29A-29F or 30A-30D may have a fluid chambers 622A and 622B or may have fluid chambers 622A, 622B, and 622C. If the device 600 has only fluid chambers 622A and 622B, then only one of the halves 604 or 606 corresponding to the fluid chamber 622A has ports 640 and 644. If the device 600 has fluid chambers 622A, 622B, and 622C, then both halves 604 and 606 have ports 640 and 644.

Nothing herein shall limit the combination of embodiments, in whole or in part, described herein.

1. A corporeal device for filtering and oxygenating blood, comprising:
an elongated tubular housing having an elongated tubular frame and a bio-modified material layer, the tubular frame coaxially expandable and collapsible relative to a central axis extending through the tubular frame, the bio-modified material layer covering at least an entire outer surface of the tubular frame, and a cavity defined by the bio-modified material layer and the tubular frame, wherein the elongated tubular housing has an opening along a longitudinal end of the tubular housing;
a first pouch connected to a first tube, the first tube extending into the cavity via the opening; and
a second pouch connected to a second tube, the second tube extending into cavity via the opening,
wherein the device is configured to create at least one differential across the bio-modified material layer between the blood outside the tubular housing and a differential matter held within the cavity, and wherein the bio-modified material layer is semi-permeable.

2. The corporeal device of claim 1, further comprising a third pouch connected to a third tube, the third tube extending into the cavity via the opening.

3. The corporeal device of claim 2, wherein the third pouch defines a third pouch cavity connected to the third tube, an entire outer surface of the third pouch and third tube covered in a third pouch bio-modified material layer.

4. The corporeal device of claim 2, wherein the first tube is shorter than the third tube by at least 15 mm (millimeters).

5. The corporeal device of claim 4, wherein the second tube is 15 mm in length.

6. The corporeal device of claim 1, wherein the first pouch defines a first pouch cavity fluidly connected to the first tube, an entire outer surface of the first pouch and the first tube covered in a first pouch bio-modified material layer; and
wherein the second pouch defines a second pouch cavity fluidly connected to the second tube, an entire outer surface of the second pouch and the second tube covered in a second pouch biomodified material layer.

7. The corporeal device of claim 1, wherein the tubular frame is made from a memory-shape material.

8. The corporeal device of claim 7, wherein the memory-shape material is nitinol.

9. A corporeal device for filtering and oxygenating blood, comprising:
an elongated tubular housing, the tubular housing being semi-rigid, deformable from an operational shape, and reformable to the operational shape, the tubular housing defining an inner cavity, wherein the inner cavity has an opening along a longitudinal end of the tubular housing;
a first pouch connected to a first tube, the first tube extending into the inner cavity via the opening; and
a second pouch connected to a second tube, the second tube extending into the inner cavity via the opening,
wherein the tubular housing has a bio-modified material layer covering at least an entire outer surface of the tubular housing, and
wherein the device is configured to create at least one differential across the bio-modified layer between the blood outside of the tubular housing and a differential matter contained within the inner cavity.

10. The corporeal device of claim 9, wherein the bio-modified material layer is semi-permeable.

11. The corporeal device of claim 10, wherein the opening is sealed around the first tube and the second tube.

12. The corporeal device of claim 11, wherein the differential matter is a gas.

13. The corporeal device of claim 12, wherein the gas contains oxygen.

14. The corporeal device of claim 11, wherein the differential matter is a fluid.

15. The corporeal device of claim 11, wherein the inner cavity, a hollow portion of the first tube, a hollow portion of the second tube, a first pocket defined by the first pouch, and a second pocket defined by the second pouch define an inner environment of the device.

16. The corporeal device of claim 15, wherein the inner environment of the device is sealed from an outer environment and is only permeable across the bio-modified material layer of the tubular housing.

17. The corporeal device of claim 9, further comprising a third pouch connected to a third tube, the third tube extending into the inner cavity via the opening.

18. The corporeal device of claim 17, wherein the third pouch defines a third pocket connected a hollow portion of the third tube, both the third pocket and the hollow portion of the third tube open to the inner cavity.

19. The corporeal device of claim 18, wherein an entire outer surface of the third pouch and the third tube are covered with a third pouch bio-modified material layer.

20. The corporeal device of claim 9, wherein an entire outer surface of the first pouch and the first tube are covered with a first pouch bio-modified material layer, and an entire outer surface of the second pouch and the second tube are covered with a second pouch bio-modified material layer.

21. The corporeal device of claim 9, wherein the device is used to perform dialysis.

22. The corporeal device of claim 9, wherein the device is used to perform cardio-pulmonary bypass.

23. The corporeal device of claim 9, wherein the device is used to perform extra-corporeal membrane oxygenation.

24. The corporeal device of claim 9, wherein the device is fully implanted with a human body.

25. The corporeal device of claim 24, wherein the elongated tubular housing is positioned within a blood vessel of the human body.

26. The corporeal device of claim 25, wherein the first tube and the second tube bisect a wall of the blood vessel.

27. The corporeal device of claim 26, wherein the first pouch and the second pouch are positioned beneath a layer of skin of the human body.

28. The corporeal device of claim 27, wherein the wall of the blood vessel is fluidly sealed around the first tube and the second tube.

29.-56. (canceled)
